(19) United States
(12) Patent Application Publication
(10) Pub. No.: US 2024/0382461 A1
(43) Pub. Date: Nov. 21, 2024

(54) PHARMACEUTICAL COMPOSITION AND METHOD FOR PREVENTION AND TREATMENT OF HEARING LOSS

(71) Applicant: PHARMAZZ, INC., Willowbrook, IL (US)

(72) Inventor: Kartike Gulati, Naperville, IL (US)

(73) Assignee: PHARMAZZ, INC., Willowbrook, IL (US)

(21) Appl. No.: 18/317,360

(22) Filed: May 15, 2023

(51) Int. Cl. A61K 31/4178; A61K 9/00; A61P 27/16

(52) U.S. Cl. CPC A61K 31/4178; A61K 9/0019; A61P 27/16

(57) ABSTRACT

The aspects of the disclosed embodiments relate to a composition using an endothelin B receptor agonist, such as sovateltide (N-Succinyl-[Glu⁹, Ala^{11,15}] endothelin 1, IRL-1620), for increasing proliferation and protecting the existing neuroepithelium of cochlear and vestibular cells located within the otic capsule. The composition is placed transtympanically, via a cochleostomy, or systemically through an intravenous or intramuscular form. A method for treating sensorineural hearing loss and vestibular symptoms caused by degeneration of inner ear cells using a compound that includes endothelin analogs by the mechanism of neuroprotection and neurogenesis of cochleovestibular hair cells and synapses.

### FIELD

The present disclosure relates to compositions and methods comprising endothelin B (ETB) receptor agonist for increasing proliferation and protecting existing neuroepithelium of cochlear and vestibular cells located within the otic capsule and related methods thereto in treating and preventing hearing loss.

### BACKGROUND

Hearing loss affects 48 million people in the United States (Center for Hearing and Communication) and 360 million people worldwide, of which 32 million are children. Two types of hearing loss that exist include conductive hearing loss and sensorineural hearing loss. Conductive hearing loss indicates there is a problem with conducting sound from the environment to the inner ear. This may include swelling in the ear canal, cerumen impaction, tympanic membrane perforation, ossicular chain abnormality, and middle ear effusion. Sensorineural hearing loss is a problem with the actual organ of hearing, which is the cochlea or the nerve synapse in the cochlea. The cochlea contains hair cells, which translate mechanical energy to neural signals. The spiral ganglia is the nerve synapse located within the cochlea. Sensorineural hearing loss accounts for 70% of cases of hearing loss (Strenzke et al. 2008).

Impairment due to sensorineural hearing loss has a significant social and economic impact because it affects the ability to interact with people and the surrounding environment and, when it occurs early in life, causes language development delays and social integration problems (Ciorba et al. 2014; Martini et al. 2014).

Sensorineural hearing loss includes ototoxicity, deafening noise, presbycusis, gene mutations, viral infections, congenital abnormalities, Meniere's disease, an autoimmune condition, iatrogenic, mass occupying lesions in the brain, vascular ischemia (Schacht et al. 2012; Seixas et al. 2012). Etiologies outside the cochlea affect the cochleovestibular nerve or more central auditory pathways. This can include pathology, such as auditory neuropathy. Etiologies within the cochlea will include problems with the neuroepithelium, including the hair cells and/or problems with hair cell synapses.

The cochlea contains hair cells, which translate mechanical energy to neural signals. The spiral ganglia is the location of the nerve synapse in the cochlea. Sensorineural hearing loss is considered permanent in cases where there is damage to the neuroepithelium within the cochlea. Humans do not show endogenous cellular regeneration in the inner ear.

Currently, no effective exogenous therapy allows the replacement of damaged hair cells or the regeneration of the spiral ganglia. Studies show that after an inner ear insult resulting in synaptic degradation or after the damage of the sensory epithelium of the cochlea with loss of neurotrophic support, spiral ganglion neurons retract peripheral neurites and slowly degenerate (Stankovic et al. 2004). Potentially transient sensorineural hearing loss may be due to Meniere's disease, labyrinthitis, Idiopathic sudden sensorineural hearing loss, and noise-induced hearing loss. If diagnosed and treated at an early stage, this type of sensorineural hearing loss may completely or partially recover. The most common treatment includes oral and/or transtympanic steroid administration (Filipo et al. 2014).

Current treatments involve amplification with hearing aids or implantable devices for profound hearing loss. No pharmacologic agent exists in the market to assist with sensorineural hearing loss by the mechanism of otic capsule neuroepithelial regeneration.

In view of the aforementioned drawbacks, there exists a need to provide a compositions comprising endothelin B (ETB) receptor agonist for increasing proliferation and protecting existing neuroepithelium of cochlear and vestibular cells located within the otic capsule, thereby preventing hearing loss.

### SUMMARY

The aspects of the disclosed embodiments are directed towards comprising a method for treating sensorineural hearing loss and vestibular symptoms caused by degeneration of inner ear cells using a compound that includes endothelin analogs by the mechanism of neuroprotection and neurogenesis of cochleovestibular hair cells and synapses. Endothelin B receptors have been found to be important in the growth and maintenance of inner ear hair cells.

The disclosed embodiments provide a method of producing an expanded population of cochlear supporting cells or vestibular supporting cells using a composition containing an endothelin analog, including an endothelin B receptor agonist (sovateltide, IRL-1620). This composition assists in expanding the population of cochleovestibular neuroepithelium by differentiation of progenitor stem cells, thereby assisting in an increased population of cells that would assist in hearing loss and balance disorders, including cochlear hair cells.

In another embodiment, the cochlear and vestibular supporting cells are mature. In such cases, endothelin analogs, including endothelin B receptor agonist (sovateltide, IRL-1620), would assist in the neuroprotection of the cochleovestibular neuroepithelium.

In another embodiment a method provides treating a subject who has, or is at risk of, developing an inner ear hearing disorder comprising administration of a composition containing endothelin analog, including endothelin B receptor agonist (sovateltide, IRL-1620).

In some embodiments, the hearing disorder is described as sensorineural hearing loss. In some embodiments, the hearing disorder is described as mixed hearing loss.

In some embodiments of the methods of the disclosure, the treatment results in improved auditory function when assessed by auditory brainstem response (ABR) testing after administering endothelin B receptor agonist sovateltide during noise trauma. In some embodiments of the methods of the disclosure, the increase of cochleovestibular neuroepithelium compared to the vehicle control is measured by an assay involving mitochondrial survival using electron microscopy. The resulting microscopy, after staining surviving mitochondria, shows significant neuroprotection of the cochleovestibular neuroepithelium compared to the vehicle control.

In some embodiments of the methods of disclosure, the endothelin B receptor agonist is administered locally and/or systemically.

In some embodiments of the methods of disclosure, the local administration is to the tympanic membrane, the middle ear or the inner ear. In some embodiments of the methods of disclosure, the local administration is to the middle ear. In some embodiments of the methods of disclosure, the systemic administration is oral or parenteral. In some embodiments of the methods of disclosure, the systemic administration is oral.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present invention, suitable methods and materials are described below. In the case of conflict, the present specification, including definitions, will control. In addition, the materials, methods, and examples are illustrative only and are not intended to be limiting.

Other features and advantages of the disclosed embodiments will be apparent from the following detailed description and claims.

### BRIEF DESCRIPTION OF THE DRAWINGS

These and other features, aspects, and advantages of the disclosed embodiments will become better understood with regard to the following description, appended claims, and accompanying drawings where:

FIGS. 1A and 1B illustrates a graphical representation of ABR thresholds at Baseline and Day 14 as a function of treatment group and frequency. Data are presented as group mean±SEM;

FIGS. 2A, 2B and 2C illustrates a graphical representation of ABR threshold shifts from Baseline to Day 14 as a function of treatment group and frequency in vehicle-treated groups (A) and after noise (B) and K/F (C) exposure; and

FIG. 3 illustrates fluorescence confocal microscopy imaging showing mitochondrial (mt) staining in serum-free and serum-containing media using JC 1 dye. A red stain shows active mitochondrial potential, whereas a green stain shows compromised mitochondrial potential.

FIG. 4 illustrates a summary of the study design where male and female mice were divided into seven groups, each with ten mice. Groups 1-3 were treated with 112-decibel noise to induce cochlear trauma, whereas groups 4-6 were treated with kanamycin+frusomide to induce aminoglycoside-induced cochlear trauma, and group 7 was kept as the control. In addition, Sovateltide in two different doses was administered in groups 1, 2, 4, and 5, while the vehicle, phosphate-buffered saline, was administered in groups 3, 6, and 7. The auditory brainstem response (ABR) test was performed at baseline and day 14 after noise or aminoglycoside exposure.

### DETAILED DESCRIPTION

The following description includes the preferred best mode of one embodiment of the present disclosure. It will be clear from this description that the disclosed embodiments are not limited to these illustrated embodiments but that the disclosed embodiments also include a variety of modifications and embodiments thereto. Therefore, the present description should be seen as illustrative and not limiting. While the disclosed embodiments are susceptible to various modifications and alternative constructions, it should be understood, that there is no intention to limit the disclosed embodiments to the specific form disclosed, but, on the contrary, the disclosed embodiments are to cover all modifications, alternative constructions, and equivalents falling within the spirit and scope of the disclosed embodiments as defined in the claims.

In any embodiment described herein, the open-ended terms “comprising,” “comprises,” and the like (which are synonymous with “including,” “having” and “characterized by”) may be replaced by the respective partially closed phrases “consisting essentially of,” consists essentially of,” and the like or the respective closed phrases “consisting of,” “consists of, the like.

As used herein, the singular forms “a,” “an,” and “the” designate both the singular and the plural, unless expressly stated to designate the singular only.

The aspects of the disclosed embodiments are directed toward method and composition to prevent and treat hearing loss using endothelin B receptor analogs such as N-Succinyl-[Glu9, Ala11,15] endothelin 1 (IRL-1620, sovateltide), BQ-3020, [Ala1,3,11,15]-endothelin, sarafotoxin S6c, and endothelin 3.

In various aspects, the aspects of the disclosed embodiments provide methods for increasing and/or protecting the neuroepithelium, including the support cells of the otic capsule, which comprises the cochlea to assist with hearing loss using Endothelin analogs.

Endothelin (ET), an endogenous 21 amino acid peptide, was first isolated from porcine aortic endothelial cells nearly 3 decades ago (Yanagisawa et al. 1988). There are 3 distinct isopeptides: ET-1, ET-2, and ET-3, which are present in various mammalian tissues performing a myriad of physiological and pathological roles such as regulation of blood pressure and perfusion, apoptosis and cellular proliferation and migration (Ehrenreich et al. 2000; Inoue et al. 1989; Vidovic et al. 2008; Yanagisawa et al. 1988). The ET peptides produce their biological effects through the activation of G-protein-coupled receptors (GPCR): ET_{A} and ET_{B} (Arai et al. 1990). Initial studies suggested two subtypes of ET_{B} receptors in the brain; ET_{B1} receptors with super high affinity and ET B2 receptors with high affinity binding to ET ligands (Sokolovsky et al. 1992). Subsequently, it was suggested that there are two subtypes of ET_{B} receptors; ET_{B1} which are IRL-1620 sensitive and ET_{B2} which are IRL-1620 insensitive receptors (Brooks et al. 1995). While assessing the role of endogenous ET it was reported that there are two subtypes of ET_{B} receptors; RES-701 sensitive mediating vasodilation, ET_{B1} receptors and RES-701 insensitive mediating vasoconstriction ET_{B2} receptors (Gellai et al. 1996; Miasiro et al. 1998). However, ET_{B} receptor subtypes have never been cloned and are not recognized as receptors (Davenport 2002) and no further Family A GPCRs have been identified that might bind ET peptides (Davenport et al. 2016).

ET-1 and its receptors, however, are not limited to the vascular system. Indeed, high concentrations of ET-1 are made by neurons, astrocytes and glial cells in the central nervous system (CNS) (MacCumber et al. 1990). ET_{A} and ET_{B} receptors in the CNS are important regulators of homeostatic conditions-regulating the sympathetic nervous system and cerebral blood flow (CBF) as well as neuronal migration, proliferation and apoptosis (Ehrenreich et al. 2000; Gulati et al. 1992; Gulati and Srimal 1993; Vidovic et al. 2008). The development and use of selective and non-selective agonists and antagonists for the ET_{A} and/or ET_{B} receptor has allowed researchers to delineate the actions of these receptors with regard to CNS development, pathogenesis and repair.

Sovateltide (IRL-1620) [N-Succinyl-[Glu9, Ala11,15] endothelin 1] is a synthetic analog of ET-1 which was synthesized in 1992 (Takai et al. 1992). The names sovateltide, PMZ-1620, SPI-1620, and IRL-1620 are synonyms with amino acid sequence Suc-Asp-Glu-Glu-Ala-Val-Tyr-Phe-Ala-His-Leu-Asp-Ile-Ile-Trp, molecular weight of 1821.9 and molecular formula C₈₆H₁₁₇N₁₇O₂₇; CAS #142569-99-1. Sovateltide (IRL-1620) is a potent and specific agonist for the ET_{B} receptors, with Kᵢ values for ET_{A} and ET_{B} receptors of 1.9 UM and 16 pM, respectively, making it ˜120,000 times more selective for ET_{B} over ET_{A} receptors. Since its synthesis, IRL-1620 has been used in numerous studies to determine the biological actions of ET_{B} receptors in the pulmonary, hepatic, renal, gastrointestinal, dermatological and endocrine systems (Bauer et al. 2000; Fellner and Arendshorst 2007; Khan et al. 2006; Lawrence et al. 1995; Mathison and Israel 1998; Mazzoni et al. 1999). We and others have used IRL-1620 to determine the role of ET_{B} receptors in the CNS (Briyal et al. 2015; Briyal et al. 2014; Gulati et al. 1997; Gulati et al. 1996; Gulati et al. 1995; Gulati et al. 2017; Kaundal et al. 2012; Leonard et al. 2011; 2012; Leonard and Gulati 2013; Leonard et al. 2015) which are present in high concentrations (Druckenbrod et al. 2008; Hostenbach et al. 2016; MacCumber et al. 1990; Schinelli 2006). The human brain contains a high density of ET receptors (Schinelli 2006), with ET_{B} accounting for 90% of total ET receptors in the cerebral cortex (Harland et al. 1995), localized to neuronal regions. The relative expression of ET_{B} receptors was found to be highest in the human cerebellum, brainstem, hypothalamus, cerebral cortex, hippocampus, striatum, olfactory bulb and lungs (Davenport et al. 2016). ET_{B} receptors were not detected in the vascular structures or leptomeninges (Davenport et al. 2016). We have found that IRL-1620 promotes neuronal cell proliferation (Gulati 2016) and the location of neuronal stem cells are predominantly in the subventricular zone (SVZ), lining the wall of the lateral ventricles, hippocampal dentate gyrus (Eriksson et al. 1998; Kuhn et al. 1996) and spinal cord (Barnabe-Heider et al. 2010) of the adult CNS. In addition, a direct contact between endothelial cells and neuronal stem cells lining the ventricles is critical for maintaining the stemness (Ottone et al. 2014). Considering these factors, it appears that the main site of action of IRL-1620 for neurogenesis will be the lining of the walls of the cerebral ventricles.

While numerous studies have been performed to determine the role of central ET_{A} receptors by pharmacologically stimulating and blocking these receptors, the role of central ET_{B} receptors has largely been ignored, and only a few studies have been conducted to explore the function of these receptors. We will focus on studies that bring insight into the functionality of ET_{B} receptors in the CNS. Studies have been carried out to determine the role of ET_{B} receptors in the development of CNS and the effect of stimulating ET_{B} receptors via selective agonist, IRL-1620, in animal models of cerebral ischemic stroke, Alzheimer's disease (AD) and hypoxic ischemic encephalopathy.

### Endothelin B Receptors in the Developing CNS

ET_{B} receptors are known to be an essential component of the developing nervous system. During both pre- and post-natal development, ET_{B} receptors help to regulate the differentiation, proliferation and migration of neurons, melanocytes and glia of both the enteric and central nervous systems (Druckenbrod et al. 2008). Serious or even fatal birth defects have been associated with pre-natal disturbances in ET_{B} receptor expression or function. The rodent ET_{B} receptor knockout model, which leads to mortality within 4 weeks of birth, is characterized by craniofacial malformation and congenital aganglionosis in the gastrointestinal tract as well as alterations in neuronal and glial cells (Dembowski et al. 2000). Within the CNS, these ET_{B} deficient rats show high levels of ET-1 with the cerebrovasculature demonstrating an enhanced constrictor response, along with an increase in apoptosis and a distinct decrease in the number of neural progenitor cells (Ehrenreich et al. 2000; Ehrenreich et al. 1999; Vidovic et al. 2008). In the early human embryo, ET_{B} mRNA expression is limited to the neural tube, sensory and sympathetic ganglia and endothelium (Brand et al. 1998). While these studies demonstrate the actions and importance of ET_{B} receptors in the pre-natal CNS, little was previously known about the role of ET_{B} in post-natal CNS development.

Post-natal rat brain ET_{B} receptor expression studies conducted in our laboratory have shown that ET_{B} expression decreases by 72% in the normal rat brain from post-natal day 1 to 28 (Puppala et al. 2015). Specifically, there was a significant decrease in ET_{B} expression in the cerebral cortex and SVZ by the 7th and 14th days of life, while expression within the cerebrovasculature increased. These results suggest that ET_{B} receptors are involved in the structural maturity and development of the CNS during post-natal period, after which the requirement of ET_{B} receptors diminishes leading to decreased expression in the CNS. Indeed, a further study found that the decrease in ET_{B} expression in the neuronal tissue coincided with a similar decrease in nerve growth factor (NGF), while administration of IRL-1620 on post-natal day 21 resulted in a significant increase in both ET_{B} and vascular endothelial growth factor (VEGF) in the cerebrovasculature (Leonard et al. 2015).

The findings that ET_{B} receptor stimulation is necessary for pre- and post-natal development and can influence growth factors like VEGF and NGF indicated that these receptors could serve as a potential target for neurovascular remodeling in the adult CNS as well. The endogenous neurorestorative processes within the adult brain attempt to repair damage due to disease, trauma or hypoxia by initiating neurogenesis, angiogenesis and oligodendrogenesis. It is possible that pharmacological interventions such as IRL-1620-induced stimulation of ET_{B} receptors can enhance these innate processes to improve neurovascular repair and remodeling or neuroregeneration.

### Endothelin B Receptors in Hearing Loss

Studies have shown endothelin (ET) has a role in sensorineural hearing loss. Within one study, ischemia appeared on the capillary of stria vascularis and the cilium of hair cell displayed significant disorder. At this time, the level of ET in plasma rose temporarily. ET_{A} and ET_{B} is expressed in the plasma of the intermediate cells and the capillary walls in the stria vascularis (Xu et al. 2005). The study concluded that severe disturbance of the cochlear circulation occurred during the course of the noise injury to the inner ear. At this time, the activity of the ET system of the cochlea stepped up significantly and had a direct relationship with ischemia of the stria vascularis. Another study showed the contents of plasma nitric oxide (NO) and ET remarkably change during the process of sudden hearing loss. The content of plasma ET in sudden hearing loss was higher than in the control group; however, the NO level was lower (Liu et al. 2003). Waardenburg-Shah syndrome is an auditory-pigmentary disorder that combines features of pigmentary abnormalities of the skin, hair and irides, and sensorineural hearing loss. A study found that mutations in the endothelin-B receptor gene on 13q22 was found to cause this syndrome (Tuysuz et al. 2009). Another study showed that endothelin could be implicated in hearing loss and tinnitus via vasoconstriction of the spiral modiolar artery, which supplies the cochlea. The study found that endothelin-1 (ET-1) induced a transient [Ca2+] increase and a strong and long-lasting vasoconstriction of the gerbil spiral modiolar artery via ETA receptors (Scherer et al. 2001). Another study tested the theory of reversing the ET-1 induced vasoconstriction of the spiral modiolar artery. It was found that Rho-kinase inhibition and cell-permeable analogue of CAMP (dbcAMP) reversed the ET-1 induced vasoconstriction along with the ET-1 induced increase in Ca2+ sensitivity (Scherer et al. 2005). It has also been shown that Lys198Asn (G/T) polymorphism of the endothelin-1 gene showed that recessive genotypes were significantly associated with increased sudden sensorineural hearing loss implicating a relationship between endothelin-1 and sudden sensorineural hearing loss (Uchida et al. 2013). It has also been disclosed in a study (Kato et al. 2011) where Dbh/Ednrb-Tg mice were administered with luteolin, and an expression of Ednrb in the lid film of the inner ear was observed. Luteolin was found to be effective against noise related and age-related hearing loss.

Another patent application relates to compounds and methods for the regeneration and/or restoration of hair cells utilizing a compound or an agent that decreases expression of a gene such as Hes1 in a tissue of the inner ear and a second agent. In some embodiments, the compound or agent that decreases expression of a gene in a tissue of the inner ear may include a siRNA. In some embodiments, this second agent is a GSK-3 inhibitor or FGF2 (West and Kopke 2017).

Another strategy is to use compounds and methods for such as Wnt agonist, GSK3-alpha inhibitor, or GSK3-beta inhibitors in combination with TGF-beta inhibitors to induce the self-renewal of stem/progenitor supporting cells, including inducing the stem/progenitor cells to proliferate while maintaining in the daughter cells the capacity to differentiate into hair cells (McLean 2017).

Treatment versions are proposed that activate the cell signaling pathway Sonic hedgehog. Vitronectin is used along with at least one anti-tumor agent or a mixture of vitronectin, and at least one glucocorticoid is used for damaged hair cell regeneration in the inner ear for sensorineural hearing loss (Zhuravlev 2017).

Double-stranded RNA (dsRNA) mols. targeting genes HES1, HES5, HEY2, CDKN1B, and NOTCH1, for RNA interference-mediated down-regulation of these genes related to hearing, balance, and inner ear hair cell growth has been described. The use of dsRNA was shown to reduce carboplatin-induced and acoustic trauma-induced hair cell death in the cochlea of chinchilla models, as well as to reduce cisplatin-induced hair cell death in the cochlea of rats. Further, dsRNA use was shown to reduce noise-induced death of otic sensory cells of the inner ear of guinea pigs and to induce hair cell regeneration in rat models of ototoxic hearing loss (Adamsky and Feinstein 2013). Combinations of inhibitors directed at the down-regulation of genes associated with hearing loss, including HES1, HES5, HEY2, CDKN1B and NOTCH1, exhibiting a beneficial effect and are useful in treating or attenuating hearing loss, treating balance impairment, promoting the replacement, and regeneration, or protection of otic (sensory) hair cells of the inner ear, and or effecting hearing restoration/regeneration has been proposed (Adamsky and Feinstein 2014).

An inner ear cell induction method to guide the inner ear stem cells from pluripotent stem cells has been described by culturing the pluripotent stem cells in the presence of ROCK inhibitor, step of culturing in the ROCK inhibitor absence, step of culturing in a serum-free medium, step of culturing in growth factor-containing serum-free medium, and step of dissociation into a single cell. The pluripotent stem cells could be embryonic stem cells or induced pluripotent stem cells (Hosoya et al. 2015).

Methods and compositions for inducing cells of the inner ear (for example, cochlear and utricular hair cells) to reenter to cell cycle and to proliferate using agents that increase c-myc activity and/or Notch activity have been proposed for treatment of hearing loss or vestibular dysfunction (Chen 2014).

Treating or preventing hearing loss by administering Myc or an agent that increases the expression of Myc in an inner ear organ has been described. The Myc family protein is selected from the group consisting of c-Myc, N-Myc, L-Myc, and any combination thereof. The agent could be an expression vector comprising a nucleic acid sequence encoding a Myc family protein and the vector could be an adenovirus vector (Burns and Jackson 2013).

Many of the above patent applications that have been filed using different strategies to promote growth or regeneration of inner ear hair cells. However, none of them propose to use endothelin B receptor analogs such as N-Succinyl-[Glu9, Ala11,15] endothelin 1 (IRL-1620, sovateltide), BQ-3020, [Ala1,3,11,15]-endothelin, sarafotoxin S6c, and endothelin 3. The present invention is directed toward method and composition to prevent and treat hearing loss using endothelin B receptor analogs such as N-Succinyl-[Glu9, Ala11,15] endothelin 1 (IRL-1620, sovateltide), BQ-3020, [Ala1,3,11,15]-endothelin, sarafotoxin S6c, and endothelin 3.

Current treatment for sensorineural hearing loss relies solely on the amplification of sound (hearing aids). Surgery has been performed to manage certain sensorineural hearing loss. This includes a bone anchored hearing device (Håkansson 2005) which is meant for single sided deafness. This hearing device is implanted in the skull on the side of hearing loss transmitting sound through bone conduction to the opposing cochlea. In severe cases of sensorineural hearing loss, electrical stimulation of remaining neurons is employed (cochlear implants) (Muller and Barr-Gillespie 2015). Cochlear implants (CIs) (Money et al. 2008) consist of inserting electrodes directly into the cochlea stimulating the spiral ganglia. In this case the device itself converts acoustic or mechanical energy (sounds) to electrical signals. Other hearing devices may be implanted in the middle ear space, typically coupling with ossicles to enhance sound conduction. Examples of this are included in the following patent(s): (Engebretson and Fredrickson 1992; Heide and Prescott 1993; Julstrom et al. 2009). These technological devices are limited by the function of the spiral ganglion neurons. All options in managing or treating sensorineural hearing loss do not fully restore a biologic hearing experience.

The ideal solution for sensorineural hearing loss would be preventing cochlear hair cell loss or regeneration of cochlear hair cells. The solution needs to include repairing or preventing damage to the spiral ganglion neurons. Stem cell (SC), gene or drug therapies are currently under investigation to solve this challenging problem.

Regenerative medicine has shown success in several research and clinical fields, such as dermatology, cardiovascular medicine, and orthopedics (Wallace et al. 2012). Among regenerative medicine strategies, the use of stem cells to restore damaged tissues is one of the most studied cell-based applications (Li et al. 2017). In otology, for example, SCs transplanted on synthetic scaffolds have recently been applied in tissue engineering for the reconstruction of the human auricle (Valente et al. 2017; Villar-Fernandez and Lopez-Escamez 2015).

Current inner ear cell regeneration includes exogenous cell transplant, endogenous induction/regeneration, gene/drug therapy, and neurotrophic factors (Almeida-Branco et al. 2015).

### Methods of Use

In certain embodiments, endothelin b agonist would be used with drug inhibition of the Notch-activated signals after ototoxic damage. In mammals, the supporting cells in the inner ear, which surround the cochlear hair cells, are capable of returning to the cell cycle, dividing and substituting the damaged cochlear hair cells. This has been shown through drug inhibition of the Notch-activated signals after ototoxic damage (Bramhall et al. 2014). These cells are all Lgr5-positive supporting cells. These Lgr5 supporting cells do not regenerate spontaneously in response to cochlear hearing loss (Rivolta 2013).

In certain embodiments, endothelin b agonist would be used in addition to exogenous administration of stem cells to assist in differentiating an increase population of cochleovestibular neuroepithelium. Research is focusing on differentiating haematopoeietic stem cells that are typically derived from the bone marrow, and have been found in adult mouse cochlea. These cells typically differentiate into macrophages and fibrocytes; however, research is being done to see if they can differentiate into cochlear stem cells (Hirose et al. 2005).

Differentiation of embryonic stem cells to a hair cell has been demonstrated by Oshima et al. with a step-wise protocol that mimics early embryonic development. Electrophysiological studies demonstrated functional stereociliary bundles and mechanosensitivity in these embryonic stem cell derived hair cells (Oshima et al. 2010). It is important to note that only 0.36% of the plated embryonic stem cells developed as hair cells.

Another strategy has been attempting to transplant stem cells into the inner ear. Based on the article by Okano et al., targets for stem cell transplantation include four directions. First is direct delivery into the organ of Corti, which would be an ideal location; however, due to its small size and inaccessibility would pose a considerable challenge. Another option is transplanting stem cells to the spiral ganglion. This could also benefit the efficacy of implantable devices such as cochlear implant. A third option is the stria vascularis and spiral ligament. Lastly, genetically modified stem cells as vectors to produce factors that promote regeneration or regrowth of specific structures within the inner ear could be used. This would be a well suited option for promoting endogenous regeneration or repair of hair cells or spiral ganglion neurons through growth factors, hormones, endocrine/paracrine factors (Okano and Kelley 2012).

In certain embodiments, endothelin b agonist would be used with certain growth factors, cytokines, formulations, and compounds having been known to possess neurogenic and neuroprotective properties in the inner ear, including ferulic acid, arctigenin, Nerve growth factor, Thyroid hormone receptors (alpha and beta), and Tbx1 gene.

In a study, gentamicin was used to induce neuronal hearing loss in mice (Gu et al. 2017) and was assessed by auditory brainstem response and distortion product atoacoustic emissions amplitude (DPOAE). It was found that ferulic acid treatment restored auditory brainstem response threshold shifts and DPOAE due to gentamicin. It was further found that neuronal stem cell survival, neurosphere formation, and differentiation increased along with neurite outgrowth and excitability of in vitro neuronal networks (Gu et al. 2017).

Other studies showed stem cell differentiation with arctigenin. In such a study, mouse cochlea was used to isolate neuronal stem cells which were cultured in vitro. The effect of arctigenin was determined on neuronal hearing loss, and it was found that arctigenin increased survival, neurosphere formation, and neuron differentiation of neuronal stem cells in the mouse cochlea. It also restored the threshold shifts of auditory brainstem response and DPOAE in gentamicin induced ototoxicity (Huang et al. 2017).

Nerve growth factor stimulated the proliferation and differentiation of neural stem cells and restored auditory brainstem response thresholds in gentamicin induced ototoxicity, also supporting its role in neuronal hearing loss (Han et al. 2017).

Thyroid hormone receptors (alpha and beta) have been shown to play a key role in the development of the nervous system (Dussault and Ruel 1987; Farsetti et al. 1991; Rodriguez-Pena et al. 1993) including mammalian inner ear (Bradley et al. 1994).

Severe inner ear defects preventing the formation of the cochlea and vestibulum have been associated with homozygous mutation of mouse homolog. Tbx1 gene appears to be involved in the formation of otic epithelial cells (Vitelli et al. 2003), and mutations in the Tbx1 gene can lead to hearing loss.

Another embodiment includes the use of endothelin b agonist with espin isoform 1. Methods for inducing functional stereocilia and stereocilia bundles in inner ear auditory hair cells comprising administering to and/or expressing in the hair cells sufficient levels of espin isoform 1 (ESPN1, also known as DFNB36) have been described. The method further includes administering to the hair cell a polynucleotide encoding ATOH1 (atonal bHLH transcription factor 1). Further, the method includes administering to the hair cell a γ-secretase inhibitor, such as DAPT (N—[N-[(3,5-Difluorophenyl) acetyl]-L-alanyl]-L-phenylglycinetert-butyl). Further provided are otic solutions for delivering a polynucleotide encoding ESPN1 or a ESPN1 polypeptide to the inner ear of a subject (Ryan and Taura 2018).

Compositions and methods for the protection and restoration of hearing have been described (Miller et al. 2010). In particular, this invention relates to treatments using neurotrophin selected from the group consisting of glial cell line-derived neurotrophic factor, brain-derived neurotrophic factor, neurotrophin-3, neurotrophin-4/5, and ciliary neurotrophic factor to facilitate the protection and re-growth of the auditory nerve and provides methods of preventing hair cell loss and the accompanying loss in hearing (Miller et al. 2010). Another embodiment consists of administering endothelin B agonist with the selected neurotrophic as described above.

One article quoted studies attempting in-vivo regeneration of hair cells using growth factors. Another embodiment consists of using endothelin b agonist with the neurotrophic growth factors that have the greatest relationship in hair cells regeneration, including neurotrophin-3 (NT-3), brain-derived neurotrophic factor (BDNF), glial-derived neurotrophic factor (GDNF), and ciliary neurotrophic factor (CNTF) (Simoni et al. 2017). NT-3, after the birth of rats and mice, was expressed higher in hair cells but generally declined postnatally, although a partial expression remained in adult mice, mainly in inner hair cells, but also in spiral ganglion neurons, glia and supporting cells (Hansen et al. 2001; Sugawara et al. 2007). BDNF was expressed in the organ of Corti during embryonic development in rats and mice (Flores-Otero and Davis). In hair cells of adult mice, the mRNA levels of GDNF was higher than those of NT-3, and CNTF was also expressed in the spiral ganglion (Stankovic and Corfas 2003).

Another embodiment includes using endothelin b agonist in addition to altering mutations of genes encoding for gap-junction proteins. The target for supporting cells therapy in sensorineural hearing loss is the cochlear lateral wall because hearing loss is also known to be caused by mutations of genes encoding for gap-junction proteins (Hirose and Liberman 2003; Kamiya et al. 2007; Nickel and Forge 2008; Spicer and Schulte 2002; Trowe et al. 2008). These molecular alterations have been studied in the stria vascularis (Nickel and Forge 2008), in otic fibrocytes (Trowe et al. 2008), and in age-related or damage induced fibrocyte degeneration in the spiral ligament (Hirose and Liberman 2003; Spicer and Schulte 2002). Kasagi et al. (2013) and Kamiya et al. (2007) reported transplanted rat BM-MSCs in the perilymph of a sensorineural hearing loss rat model with a lateral wall fibrocyte dysfunction not associated with changes in the organ of Corti (Kamiya et al. 2007; Kasagi et al. 2013). Transplanted cells found in the injured area could express connexin 26 and connexin 30, indicating a reactivation of gap junction between neighboring cells. Moreover, the transplanted rat group showed a higher hearing recovery ratio than controls (Kamiya et al. 2007).

Another embodiment combines endothelin b agonist with Auris Medical's compound AM-111 which is serving as an exogenous therapy which contains brimapitide, or D-JNKI-1, an inhibitor of the JNK stress kinase coupled to an intracellular transporter. AM-111 is formulated in a biocompatible and fully biodegradable gel and administered in a single dose intratympanic injection into the middle ear. AM-111 blocks cell death (apoptosis) in stress-injured sensory cells and attenuates inflammation after acute inner ear injury.

Given the important role neurotrophins play in the development and maintenance of spiral ganglion neurons and other afferent connections in the peripheral auditory system, there has been a trend to try and overcome the limitations of cochlear implants by combining exogenous neurotrophin delivery with electrical stimulation from cochlear implant electrodes in animal models. The rationale behind this is to preserve spiral ganglion neuron populations so that there are more target neurons that can be stimulated. Moreover, the potential to promote the growth of afferent radial nerve fibers from the spiral ganglion neuron is appealing since it would allow for an improved electrode-neural interface (Sameer Mallick et al. 2013).

An alternative embodiment includes using endothelin b agonist with insulin-like growth factor 1 and/or hepatocyte growth factor. Another novel therapy for hearing loss under development includes utilizing sustained delivery of recombinant human insulin-like growth factor 1 (rhiGF-1) into the cochlear fluid via a biodegradable hydrogel after the onset of noise-induced hearing loss (Lee et al. 2007). This method has shown some efficacy in preventing noise induced hearing loss. Results did show the survival of outer hair cells. Another study has shown applying hepatocyte growth factor locally using biodegradable gelatin hydrogels helps attenuate noise-induced hearing loss in guinea pigs (Inaoka et al. 2009).

Another embodiment uses endothelin b agonist with a gene editing technique CRISPR-Cas9 in removing bad genes that cause hearing loss. About half of all cases of deafness are caused by genetic defects. Gene-editing technique CRISPR-Cas9 is being examined closely for its potential to prevent hearing loss in people who inherit those genes. Howard Hughes team took the approach that allows Cas9 to cut out the bad gene and then disappear before the good gene is damaged and found that in mice with mutated Tmc1 genes, after eight weeks, the inner-ear hair cells resembled those that would be present in normal animals and that treated mice could hear sounds that were 15 decibels softer than what untreated animals were able to detect. Yale scientists described an alternative gene-editing technique they developed called eMAGE (eukaryotic multiplex genome engineering), which they believe will allow new genes to be inserted into DNA without multiple double-strand breaks. Broad Institute scientists developed a system called REPAIR (RNA Editing for Programmable A to I Replacement), which targets RNA instead. They believe that may make it possible to fix gene mutations without permanently changing the genome.

In any of the above embodiments of the methods disclosed herein, the auditory disease is selected from the group consisting of sensorineural hearing loss, noise-induced hearing loss, sudden sensorineural hearing loss, autoimmune inner ear disease, tinnitus, medication-induced ototoxicity protection, radiation-induced ototoxicity protection, Meniere's disease, cranial nerve schwannoma, auditory neuropathy, cochleovestibular nerve compression syndrome, cochleovestibular nerve hypoplasia, connexin 26 mutation, genetic, congenital, and iatrogenic-related hearing loss.

In any of the above embodiments disclosed herein, the disease process treated may be beyond auditory disorders such as treating dysequalibrium, vertigo, tinnitus, fluctuations in hearing, aural fullness, vestibular neuritis, and labyrinthitis. The hearing loss treated may include any frequency and can include any degree of frequency loss measured in decibels.

### Definitions

As used herein, the term “an amount sufficient to” refers to the amount that enables the achievement of the intended effect, for example, to decrease the expression of a gene in a tissue of the inner ear. Such an amount may be determined through various assays known in the art based on the intended effect. As used herein, the terms “applying” or “administering” refer to all means of introducing the specified agent, composition, or force to the specified region or subject. “Administration” or “application” can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined, and the method of determining the most effective route of administration are known to those of skill in the art and will vary with the composition used for treatment, the purpose of the treatment, the health condition or disease stage of the subject being treated, and target cell or tissue. Non-limiting examples of routes of administration include oral administration, nasal administration, inhalation, injection, and topical application. Administration can be for use in industrial as well as therapeutic applications. As used herein the term “biodegradable” is used herein to describe substances, such as polymers, compositions, and formulations, intended to degrade during use. Biodegradable substances may also be “biocompatible,” i.e., not harmful to living tissue. Non-limiting exemplary biodegradable substances include poly(lactic acid) (PLA) and poly(lactic-coglycolic) acid (PLGA), optionally pegylated.

The term “neuroepithelium” refers to hair cells, spiral ganglion neurons, scarpas ganglion, cochleovestibular nerve, central auditory pathway, and synapse between the hair cell and its associated neurons. The term “hair cells” refer to sensory epithelial cells characterized by having long cilia (e.g., stereocilia and/or kinocilia) which appear as fine hairs under microscopy; as used herein, hair cells (HCs) may be identified by their location—e.g., inner ear hair cells (IHCs) or outer ear hair cells (OHCs) or cochleovestibular hair cells. Such hair cells are known to be present in at least the cochlear organ of Corti, maculae, and cristae of the ear. As used herein, the term “differentiation” refers to the specific conditions that cause cells to develop into cells of a mature/specialized cell type (e.g., hair cells) that produce specific gene products which coincide with and/or promote/sustain the traits of the specified mature/specialized cell type.

As used herein, the term “tissue” refers to tissue of a living or deceased organism or any tissue derived from or designed to mimic a living or deceased organism.

As used herein, the term “therapeutically effective amount” refers to a quantity sufficient to achieve a desired effect. In the context of therapeutic applications, the effective amount will depend on the type and severity of the condition at issue and the characteristics of the individual subject, such as general health, age, sex, body weight, and tolerance to pharmaceutical compositions. The skilled artisan will be able to determine appropriate amounts depending on these and other factors. In the case of an in vitro application, in some embodiments the effective amount will depend on the size and nature of the application in question. It will also depend on the nature and sensitivity of the in vitro target and the methods in use. The skilled artisan will be able to determine the effective amount based on these and other considerations. The effective amount may comprise one or more administrations of a composition depending on the embodiment. The dose range of N-Succinyl-[Glu9, Ala11,15] endothelin 1 (IRL-1620, sovateltide) could be from 0.00001 to about 1 mg and may be administered once or multiple times in a day or in weeks or in months.

As used herein, the term “treating” or “treatment” includes preventing a disease, disorder or condition from occurring in a subject predisposed to or having a disease, disorder and/or condition; inhibiting the disease, disorder or condition, e.g., impeding its progress; and relieving or reversing the disease, disorder, or condition, e.g., causing regression of the disease, disorder and/or condition. Treating a disease or condition may also include ameliorating at least one symptom of the particular disease or condition. The term “hearing loss” refers to impairment in the ability to apprehend sound; thus, treatment thereof implies any one of the above listed effects on the ability to apprehend sound. The term “sensorineural hearing loss” refers to a specific type of hearing loss where there is damage to the inner ear or to the nerve pathways from the inner ear to the brain.

### Modes of Carrying Out the Disclosure

Aspects of the disclosure relate to methods of treating hearing loss, optionally sensorineural hearing loss, and/or replacing, regenerating, or protecting hair cells through the application of one or more agents or compositions to a specified tissue or area of the ear. Areas of the ear that may be treated based on the methods disclosed herein include but are not limited to the outer, middle, or inner ear regions.

### Compositions

Aspects of the disclosure relate to a composition or an agent that regenerates and/or protects inner ear tissue neuroepithelium. The basic composition includes Endothelin B receptor agonist with or without stem cells. In some embodiments, the basic composition may not include stem cells.

In some embodiments, the basic composition may be combined with growth factors and/or cytokines known to promote regeneration and/or protection to the inner ear tissue neuroepithelium. These neuroregenerative/neuroprotective compounds include, but are not limited to, growth factors, osmotic agent such as glycerol, diuretic such as furosemide or ethacrynic acid, a steroid, transplanted stem cells, ferulic acid, arctigenin, espin isoform 1 polypeptide, neurotrophin selected from the group consisting of glial cell line-derived neurotrophic factor, brain-derived neurotrophic factor, neurotrophin-3, neurotrophin-4/5, nerve growth factor, vascular endothelial growth factor, and ciliary neurotrophic factor, insulin-like growth factor, Netrin1, thyroid hormone, hepatocyte growth factor, and luteolin.

In some embodiments, stem cells used in the basic composition may include but are not limited to haematopoetic stem cells and embryonic stem cells, and stem cells from platelet rich plasma. In some embodiments, stem cells may also be genetically modified as vectors to produce factors that promote regeneration and/or protection of inner ear tissue neuroepithelium. In some embodiments, stem cells may be transplanted into the inner ear prior to administration of the Endothelin B receptor agonist.

In some embodiments, the basic composition may be combined with compounds that have an antiapoptotic activity that allow for neuroprotection of the inner ear tissue neuroepithelium. An example is compounds that inhibit Notch-activated signals after ototoxic damage that lead to a cascade of events causing inner ear tissue neuroepithelial damage.

### Formulations

The formulations that include the basic composition that regenerates and/or protects inner ear tissue neuroepithelium is referred to as Endothelin B receptor agonist with or without stem cells.

In some embodiments, the basic composition that regenerates and/or protects inner ear tissue neuroepithelium may be synthesized in varying formulations and/or particles, as described below.

Non-limiting examples of such formulations and/or particles include a nanoparticle, lipofection, gel or hydrogel (El Kechai et al. 2016), nanoemulsion (West and Kopke 2017), microparticle (Yang et al. 2012), colloidal suspension (Ariana et al. 2016), sterile suspension (e.g., Ciprodex at http://www.ciprodex.com/), solution (Parra et al. 2002), aerosol (Li et al. 2013), powder (e.g., http://fauquierent.blogspot.com/2009/10/treatment-of-chronic-drainingear, html#ixzz459wcRKOr), eardrop (Winterstein et al. 2013), nanofiber (Akiyama et al. 2013), or cream (e.g., Quadiderm® cream). All references cited herein above are incorporated by reference in their entirety. In some embodiments, the formulations and/or particles are specifically adapted for delivery to the inner ear. For example, a gel formulation such as a thermo-reversible hydrogel (e.g., Pluronic F-127) allows for drugs to be maintained in the middle ear, in-contact with the round window membrane, such that the drug could diffuse or be transported into the inner ear. Therapeutic compound that are inner ear specific, and have been the direct target of the subject's cochlea include but are not limited to ciprofloxacin, gacyclidine, dexamethasone, hydrocortisone, methylprednisolone directly into the cochlea of the subject (Borenstein et al. 2017). A colloidal suspension could likewise be formulated specifically for injection directly into the inner ear or across the tympanic membrane for diffusion or other means of transport through the round window membrane. Likewise, a nanoparticle or formulation comprising a plurality of nanoparticles can be formulated for controlled delivery, for example, of magnetic force. Such a method can be generalized to microparticles and/or alternate nanoscale structures.

In some embodiments, the formulation may comprise a second agent in the same or different formulation and/or to facilitate the timing of its application to the target tissue simultaneous or sequential relative to the particle, the agent that regenerates or protects inner ear tissue neuroepithelium. For example, for simultaneous delivery but sequential release, the second agent may be comprised in a solution that is administered along with a sustained release formulation and/or particle comprising the agent that regenerates or protects inner ear tissue neuroepithelium. A similar effect may be achieved through the use of a single formulation and/or particle comprising both agents formulated for different release profiles for the different agents.

In some embodiments, the formulation comprises a biodegradable polymer. In further embodiments, the biodegradable polymer is poly(lactic-co-glycolic acid) (PLGA) or pegylated PLGA (PEG-PLGA). In some embodiments, the composition may include further additives, including but not limited to polyvinyl alcohol (PVA) or other known nanoparticle stabilizers. In some embodiments, the nanoparticle is magnetically responsive or includes a magnetically responsive particle. In some embodiments, the magnetically responsive particle is iron oxide, optionally superpararmagnetic iron oxide (SPION). In some embodiments, the nanoparticle may be further comprised in a solution, suspension, gel, or other formulation suitable for its delivery.

The embodiment may be formulated to facilitate the timing of release; for example, a poorly water soluble second agent (e.g., TIDE) could be encapsulated in the organic shell of ETBRA with or without stem cells loaded with the hydrophilic agent that regenerates and/or protects inner ear tissue neuroepithelium. As the second agent will be more accessible to water, it will be released first, followed by the sustained release of the agent that regenerates or protects inner ear tissue neuroepithelium.

### Modes of Administration

The above disclosed agents, compositions, formulations, and/or particles can be administered simultaneously or sequentially—with the second agent being administered before or after the agent that regenerates and/or protects the inner ear tissue neuroepithelium.

Current drug delivery methods to the inner ear include diffusion across the round window membrane, direct infusion via cochleostomy, or systemic delivery (Swan et al. 2008). Diffusion across the round window membrane will include delivery of a compound into the middle ear as the round window membrane's location serves as semi-permeable barrier between the middle ear and inner ear. Studies showed that 6-FAM-Zol, a fluorescein-BP conjugate (Kang et al. 2015; Roelofs et al. 2010), readily enters the mammalian cochlea through the round window membrane and labels the modiolus and osseous spiral lamina, the location of spiral ganglion neurons (Quesnel et al. 2012; Sun et al. 2016).

In some embodiments, application and/or administration may be, for example, direct application, injection, or infusion of a specified agent or composition. In some embodiments, the specified agent or composition can be administered by direct injection through the round window membrane or by infusion through a temporary or permanent cannula placed through the round window membrane. In some embodiments, the infusion or injection can be assisted through an attached microinfusion pump, dialysis apparatus, or fluid exchange system. In similar embodiments, injection or infusion technology could also be applied to the oval window and/or the oval window ligament or annulus. The injections or infusion could further be accomplished through a cochleostomy or other opening into the boney labyrinth, such as one of the semicircular canals. Alternatively, the cortical bone could be removed over the labyrinth, and the specified agent or composition could be applied over the decorticated bone for intraosseous delivery. In some embodiments, the composition or agent is delivered systemically through intravenous or intraarterial administration.

The above listed routes of administration are by no means exhaustive. In general, there are a variety of means of delivery to the inner ear—that fall into two general categories: through an ostomy into the inner ear (where necessary, opened by drill, knife, or laser) and through diffusion through the round window membrane, the ligament of the stapes footplate or through an area of cochlear, or vestibular structure (typically where a region of bone was thinned to a thickness so that there is only the very thinnest of bone remaining separating the middle ear space form the inner ear endosteal lining and fluid).

In some embodiments, where an ostomy is used, the ostomy is conducted by machine or by hand. In some embodiments, the ostomy is through the footplate of the stapes, through an opening drilled into the cochlea, through an opening drilled into the semicircular canal, through the vestibular aqueduct, through a cochleostomy, through a direct opening into the round window membrane. In some embodiments, the ostomy is made to insert an electrode for implantation; thus, one or more of the disclosed formulations and/or particles may be bonded to the electrode surface to elute one or more agents or compositions or formulations into the environment. In some embodiments, one or more openings subject to ostomy may be accessible for between about one day to about one week, two weeks, three weeks, four weeks, or a month, e.g. between about I to 30 days. In some embodiments, the ostomy is suited for a single injection or continuous infusion over the duration that the opening is accessible. In some embodiments, where diffusion is employed, the agents, compositions, formulations, and/or particles allow diffusion across a particular membranous structure into the inner ear fluids. Non-limiting exemplary formulations include solution, gel, emulsion, or suspension. For example, a gel or pellet may be suited for the delivery of one or more agents, compositions, and/or particles disclosed herein above. A gel, for instance, may be placed transtympanically over the stapes and over the round window membrane and over the area of thinned bone to enhance delivery by increasing the surface area for delivery. Similarly, a solid or semi-solid pellet may be placed onto the stapes footplate, round window membrane or area of thinned bone as a means of enhancing drug contact with said membranes and keeping the drug from being removed from the middle ear space.

Not to be bound by theory, one of the challenges of a less invasive diffusion approach to delivering drugs to the inner ear fluids may be the small surface area of the round window membrane and the even smaller surface area of the ligament of the stapes footplate. In some embodiments, a procedure known in the art as “blue-lining” may resolve this issue. By “blue-lining,” the drilled-out area is extremely thinned out and just barely covers the endosteal membrane on the inner surface of the inner ear. This may greatly increase the surface area for absorption and may be less invasive than making an actual opening into the cochlea or other regions of the inner ear. A skilled ear surgeon should be able to perform this procedure safely.

In some embodiments, the delivery may be achieved in single or multiple injections across the tympanic membrane. In some embodiments, the delivery may be achieved through single or multiple injections through a plastic tube inserted into the tympanic membrane. In some embodiments, the delivery may be achieved through continuous infusion through a catheter, wherein its tip is placed directly on the area where diffusion is to occur.

In some embodiments, the delivery may be achieved in a single administration directly into the endolymphatic sac located along the posterior fossa dura, whether through an incision allowing direct administration into the sac or through topical administration on the sac to allow for diffusion.

In the above instances mentioned for drug administration/delivery, the basic composition can be combined with 6-FAM-Zol, which is a fluorescein-BP conjugate that is known to readily enter the round window membrane.

In any of the above embodiments, testing will be needed to quantify regeneration and/or protection of inner ear tissue neuroepithelium. Auditory brainstem response in any of its forms may be used to test the hearing objectively along its common frequencies, which may include but are not limited to 250 Hz to 8000 Hz, and to measure the decibel of hearing capacity within each frequency. Another test to directly measure hair cell function include DPOAE (Distortion product otoacoustic emissions). Electron microscopy, in vitro measure of the neuroepithelium, etc., may also be used.

### Example 1

### Animal Study

The study aimed to determine whether Sovateltide (IRL-1620, PMZ-1620); an endothelin-B receptor agonist has otoprotective efficacy and that Sovateltide can ameliorate noise- or aminoglycoside-induced hearing loss in a mouse model system. Animals received either unilateral intra-tympanic Sovateltide at one of two dose concentrations or vehicle control injection. The study conducted unilateral auditory brainstem response (ABR) hearing tests at baseline and again at least 14 days after noise or aminoglycoside exposure as the primary measurement. A gross anatomical examination of treated middle ears was also conducted at necropsy.

### Experimental Design

| | Ototoxin/Noise | | Number of | Dose |
|---|---|---|---|---|
| Group | Treatment | Unilateral Treatment | Animals | Volume |
| 1 | noise | Dosage #1 −5 μg/kg | n = 10 | 5 uL |
| | | (25 ug/mL)Sovateltide | (5 male, | |
| | | (IRL-1620, PMZ-1620) | 5 female) | |
| 2 | noise | Dosage #2 15 μg/kg | n = 10 | 5 uL |
| | | (75 ug/mL) Sovateltide | (4 male, | |
| | | (IRL-1620, PMZ-1620) | 6 female) | |
| 3 | noise | Vehicle | n = 10 | 5 uL |
| | | (Phosphate-Buffered | (6 male, | |
| | | Saline) | 4 female) | |
| 4 | aminoglycoside | Dosage #1 5 μg/kg | n = 10 | 5 uL |
| | (kanamycin) | Sovateltide | (5 male, | |
| | | (IRL-1620, PMZ-1620) | 5 female) | |
| 5 | aminoglycoside | Dosage #2 15 μg/kg | n = 10 | 5 uL |
| | (kanamycin) | Sovateltide (IRL-1620, | (5 male, | |
| | | PMZ-1620) | 5 female) | |
| 6 | aminoglycoside | Vehicle | n = 7 | 5 uL |
| | (kanamycin) | (Phosphate-Buffered | (1 male, | |
| | | Saline) | 6 female) | |
| 7 | none | Vehicle | n = 10 | 5 uL |
| | | (Phosphate-Buffered | (5 male, | |
| | | Saline) | 5 female) | |

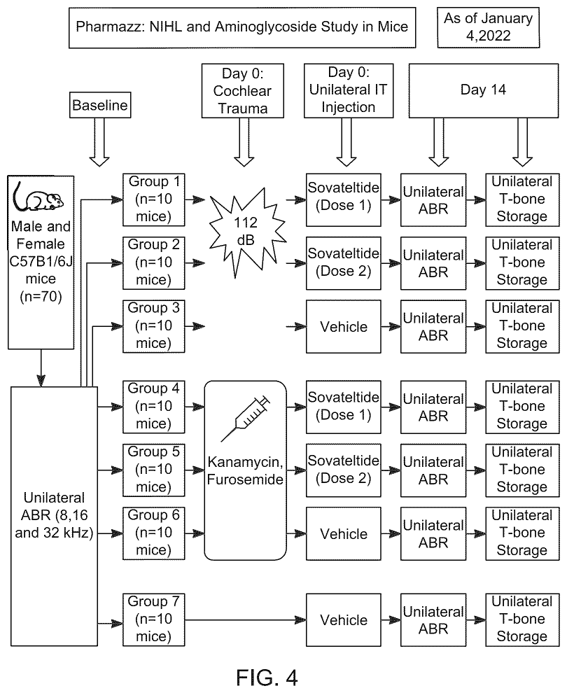

FIG. 4 describes the study design where male and female mice were divided into seven groups, each with ten mice. Groups 1-3 were treated with 112-decibel noise to induce cochlear trauma, whereas groups 4-6 were treated with kanamycin+frusomide to induce aminoglycoside-induced cochlear trauma, and group 7 was kept as the control. In addition, Sovateltide in two different doses was administered in groups 1, 2, 4, and 5, while the vehicle, phosphate-buffered saline, was administered in groups 3, 6, and 7. The auditory brainstem response (ABR) test was performed at baseline and day 14 after noise or aminoglycoside exposure.

### Study Protocol

[Male & female C57Bl/6 mice used in this study were obtained from Charles River Labs. All study animals (n=69) underwent anesthesia via 87.5 mg/kg (Ketamine)/12.5 mg/kg (Xylazine) IP injection and baseline unilateral left ear ABR testing at 8, 16, and 32 kHz.

Study animals in Groups 1-6 (n=59) underwent cochlear trauma, either via noise (112 dB SPL, 8-16 kHz band, 2-hr duration) exposure while awake, with the goal of producing a moderate chronic hearing loss in all animals or a single injection of subcutaneous kanamycin and intraperitoneal furosemide on Day 0. The noise treatment typically produced moderate hearing losses in the range of 20-50 dB and scattered losses of outer hair cells but little, if any, loss of inner hair cells. This kanamycin/furosemide treatment typically produced similar 30-50 dB threshold elevations and widespread loss of outer hair cells, with little, if any, loss of inner hair cells. Sometimes the outer hair cell loss could be nearly complete, but the exact losses in any particular study could be difficult to predict a priori.

Study animals were then divided into the following seven groups:
Group 1: noise exposure, then unilateral left side IT injection of Sovateltide at concentration 5 ug/kg in phosphate-buffered saline.
Group 2: noise exposure, then unilateral left* side IT injection of Sovateltide at concentration 15 ug/kg in phosphate-buffered saline.
Group 3: noise exposure, then unilateral left* side IT injection of phosphate-buffered saline vehicle.
Group 4: kanamycin and furosemide immediately after unilateral left* side IT injection of Sovateltide at concentration 5 ug/kg in phosphate-buffered saline.
Group 5: kanamycin and furosemide immediately after unilateral left* side IT injection of Sovateltide at concentration 15 ug/kg in phosphate-buffered saline.
Group 6: kanamycin and furosemide immediately after unilateral left side IT injection of phosphate-buffered saline vehicle.
Group 7: unilateral left* side IT injection of phosphate-buffered saline vehicle.

On post-IT injection Day 14, all study animals underwent repeat anesthesia via 87.5 mg/kg (Ketamine)/12.5 mg/kg (Xylazine) and unilateral ABR testing at 8, 16, and 32 KHz.

### Day 14 Histological/Harvesting Endpoints:

1. All injections were performed in the left ear. Subsequently, study animals were sacrificed on this day with left-sided temporal bone harvesting.
2. Left-sided temporal bones were preserved and stored temporarily for possible analysis in the future by cytocochleogram.

### Dose Administration

Frequency: Once, unilaterally, intra-tympanic, 5 mL/ear, 1 mL/second rate.

Process: The left ear of each animal received one IT placement of test article Sovateltide (IRL-1620, PMZ-1620). After injection, mice were held with their injected ear up and nose up at ˜30 degree angle to allow maximal exposure to cochlear structures.

### Mortality/Moribundity Observations

Frequency: Daily

Process: In-cage observations were made daily during the in-life study period to assess general health, moribundity, or mortality. Four mice in Group 4 and 8 mice in Group 6 were replaced due to either death or moribund condition after the combination of ketamine/xylazine 30 minutes after Furosemide and Kanamycin.

### Noise Exposure Procedure

Frequency: Once, for 120 minutes for Groups 1-3 only.

Process: Male and female mice (n=30) were given the following noise exposure: 8-16 khz, octave band-pass signal, 110-112 dB SPL, 120-minute duration, in a free field, while awake.

### Intra-Tympanic Injection of Sovateltide (IRL-1620, PMZ-1620) in the Mouse

Frequency: Once, unilaterally

Process: For IT injections, after anesthesia with ketamine/xylazine (all animals in groups 1, 2, 3, and half animals in group 7) or isoflurane (all animals in groups 4, 5, 6, and half animals in group 7), the head was positioned on the side and angled to allow otoscopic viewing/photos/videos of the ear canal and tympanic membrane (TM). The nose was elevated toward the ceiling creating a head angle with the floor of approximately 30 degrees. Injections were carried out with the assistance of a surgical microscope. Cold test article Sovateltide (IRL-1620, PMZ-1620) or vehicle was delivered at a rate of 1 ml/second through a 27 g needle fitted to a Hamilton microsyringe and delivered automatically with the assistance of a World Precision Instruments microinjection pump assembly.

A single application of drug solution was applied at one of two concentrations (5 and 15 ug/kg: mg lyophilized Sovateltide/mL phosphate-buffered saline (PBS).

Any cerumen or other debris was removed manually WITHOUT the use of alcohol, saline, sterile water, carbamide peroxide, or any other chemical agents that might irritate the skin surface or interfere/interact with Sovateltide (IRL-1620, PMZ-1620). Injections were made in an appropriate visible location through the TM, caudal to the malleus, when possible. The injection needle was bent 90° to allow for a clear view of the TM and filed to allow for the creation of a clean perforation of the TM while not so long as to damage underlying structures. Before use, the blunted needle was first hot bead sterilized for at least 30 seconds and then cooled to room temperature before use. After injection, the animal's head was then held in place for 15 minutes in the same 30-degree orientation as used in the injection (Salt et al., 20116).

### Ototoxic Agents Administration

Frequency: (kanamycin) Once, subcutaneous injection immediately after IT injections of Groups 4-6 only.

Frequency: (Furosemide) Once, intraperitoneal injection 30 minutes after Kanamycin injection to Groups 4-6 only.

### Process:

Male and female mice (n=30) were given Kanamycin (1000 mg/kg) and Furosemide (400 mg/kg) delivered 30 minutes after Kanamycin. Injections were performed according to standard SOPs¹.

### Necropsy

Euthanasia: At an assigned terminal timepoint or if considered moribund, animals would be euthanized following standard SOPs¹ and in accordance with the AVMA Panel on Euthanasia².

Unscheduled Sacrifices and Death: For any animal found dead or moribund, then euthanized, a gross macroscopic post-mortem examination would be conducted by a veterinarian or by a designated person as soon as possible. If the post-mortem examination could not be performed within 2 hours, the study animal might be stored refrigerated (2° C. to 8° C.) until an examination could be performed.

### ABR Threshold at Baseline and Day 14

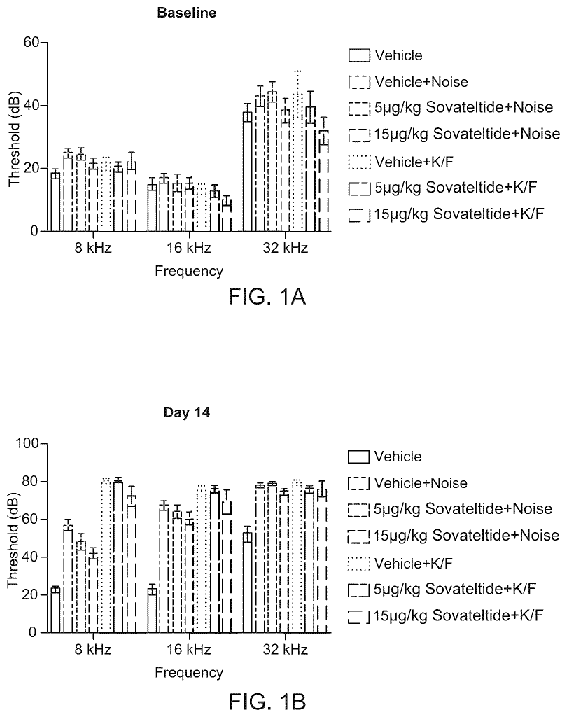

At baseline, ABR thresholds were not significantly different between groups across all 3 frequencies, suggesting similar ABR response across all groups before any drug and noise exposure (FIG. 1A). However, on Day 14 (FIG. 1B), the significant main effect of treatment (F_{(6, 60)}=32.04, p<0.0001) and frequency (F_{(2, 120)}=107.4, p<0.0001), as well as interaction (F_{(12, 120)}=16.92, p<0.0001) were observed, suggesting altered ABR response between groups at 14 days following drug and noise exposure.

ABR Threshold Shift from Baseline to Day 14

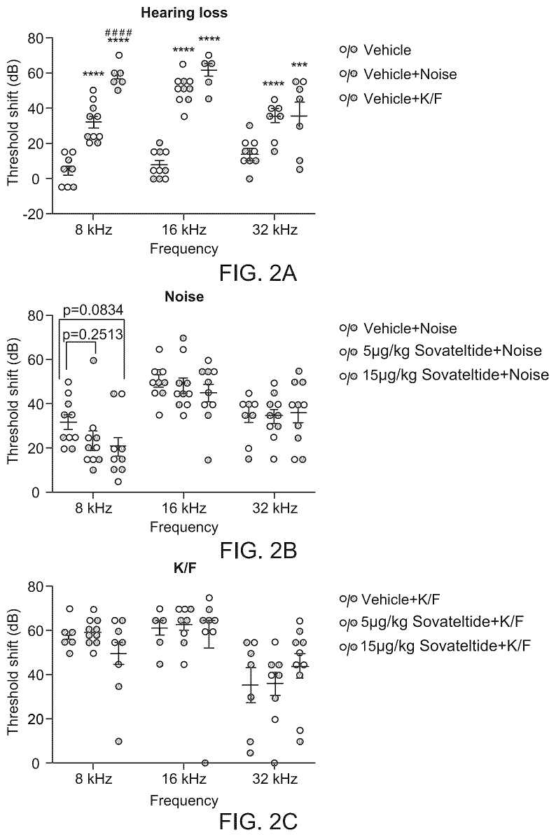

To decifer the cause of different ABR response at Day 14, follow-up hearing loss analyses were conducted on the ABR threshold shifts from baseline to Day 14 (FIG. 2). First, we checked hearing loss induced by noise exposure and K/F treatment in vehicle-treated animals (FIG. 2A). Two-way mixed model ANOVA revealed a significant main effect of treatment (F_{(2, 24)}=76.47, p<0.0001) and frequency (F_{(2, 48)}=13.38, p<0.0001), as well as interaction (F_{(4, 48)}=13.06, p<0.0001). Post-hoc tests indicated significantly higher ABR threshold shifts across all 3 frequencies in noise and K/F exposed mice, suggesting clear hearing loss induced by noise and K/F (p<0.0001 or p<0.001). In addition, compared to noise, K/F induced significantly higher ABR threshold shifts at 8 kHz (p<0.0001) and a trend at 16 kHz (p=0.0797). Next, we examined the effect of the test article, Sovateltide, on hearing loss induced by noise (FIG. 2B) and K/F (FIG. 2C). ABR threshold shifts were not significantly different between vehicle and Sovateltide groups at any test frequency after either noise or K/F exposure, suggesting no effect of Sovateltide on noise- and drug-induced hearing loss. However, there was a trend of lower threshold shift at 8 kHz with 5 (p=0.2513) and 15 (p=0.0834) ug/kg Sovateltide treatment after noise exposure (FIG. 2B). Qualitative analysis of the sex-specific response to hearing loss and Sovateltide treatment did not reveal clear sex differences, as evidenced by no obvious clustering of the open (male) and filled (female) circles (FIG. 2).

### CONCLUSION

Treatment with Sovateltide, relative to vehicle control, did not produce statistically reliable differences in hearing threshold shifts at any test frequency for either noise or aminoglycoside-induced hearing loss. However, there was a trend for treatment with 5 μg/kg and a nearly significant effect for 15 μg/kg Sovateltide to result in a better hearing at 8 kHz after noise exposure. A qualitative exploration of sex-specific effects suggests no obvious difference in males' and females' response to noise loss and Sovateltide treatment.

Based on the result from this study, at the doses tested, Sovateltide tended (though not statistically significantly) to attenuate noise-induced hearing loss at the lowest frequency tested but did not have any discernible impact on kanamycin/furosemide-induced hearing loss. Follow-up studies should explore the potential impacts on kanamycin/furosemide induced damage using a less robust damage model and noise-induced hearing loss effects could further explore sex effects, multiple dosing regimens, and/or dose concentration. Given the trends for improvement at the lowest frequency, histological evaluation of cochlear hair cells may be helpful to determine more subtle treatment effects on cochlear hair cells and/or impacts on synaptic connections between hair cells and spiral ganglion neurons.

### Example 2

Culture and treatment of adult mouse cochlear cells with sovateltide

### Methods and Materials

Inner ears comprising cochlear neuroepithelium from adult mice were dissected after anesthetizing with urethane 1.5 g/kg body weight and euthanizing with cervical dislocation. Inner ears were placed in cold saline, and the cochlea was dissected under the dissection microscope. The isolated cochlea was placed in a cold saline solution and chopped finely in a sterile condition inside a culture hood. Minced tissues were transferred to a 15 ml falcon tube and centrifuged at 1000 RPM for 2 min. The supernatant was discarded, and the pellet was submersed in 1× Trypsin EDTA solution (Cat No. R-001-100, GIBCO, MA) and triturated using a 1 ml pipette and cut-tip. The triturated tissue was incubated at 37° C. for 20 min. After 20 min, the digested tissues were vortexed gently and checked for proper digestion of the tissues. The digestion step was repeated by adding the same volume of 1× trypsin EDTA solution and incubating for 10 minutes. After a satisfactory level of digestion, the tissue solution was filtered with a sieve of pore size 40 μm to isolate cell suspension and remove undigested tissues. The cell suspension was collected in a 15 ml falcon tube, and 1 ml of serum-containing cell culture media was added and mixed properly to stop further digestion. The cochlear cell solution was centrifuged at 2000 RPM for 3 min at RT. The cell pellet was resuspended in 8 ml of 10% fetal bovine serum containing culture media and plated in 60 mm tissue culture-treated plastic plates (Alkali Scientific Inc. Fort Lauderdale, FL). The Neurobasal culture media supplemented with B-27 (Gibco, Waltham, MA) was used for culturing cochlear cells. Plates were incubated in an incubator at 37° C. with 5% CO2 for 2 weeks. Media was changed every 4^{th} day. For changing media, media with nonadherent cells were collected in a sterile falcon tube, and the plate was replenished with 8 ml of fresh media. The media containing suspended cells were centrifuged at 2000 RPM for 2 min at RT, and the supernatant was discarded. The cochlear cell pellet was resuspended in 2 ml of fresh media and seeded back into the plate. Cochlear cell attachment and spreading to tissue culture plates were observed after 1 week of culture. After 2 weeks, plates with attached cells were washed with fresh media after discarding nonadherent cells, and fresh media was replenished to allow them to grow. After cells became sufficiently confluent (3-4 weeks), they were trypsinized and plated in two new 60 mm culture plates for further culture or in a 24 wells glass bottom plate for characterization and treatment with sovateltide (IRL-1620). The characterization of cultured cochlear cells and their treatment with sovateltide (IRL-1620) was carried out as follows—

For characterization, the cells were fixed with 8% paraformaldehyde for 30 minutes and permeabilized with 1% triton X100 solution for 20 minutes at RT. Blocking was done with 4% BSA solution prepared in 1×HBSS (Hank's Balanced Salt Solution). Cells were incubated with 1:100 diluted antibodies for cochlear cell markers GDF10 and Myosin VIIa at 4° C. overnight. After washing, cells were incubated with 1:400 diluted fluorophore-tagged secondary antibody. After washing, the cells were covered with DAPI containing mounting media, and confocal microscopy using Nikon A1R Laser Scanning Confocal Microscope (Nikon, Tokyo, JP) was carried out to acquire images.

Sovateltide treatment—The cultured mouse cochlear cells were treated with 1 ng/ml sovateltide for 7 days in 2 different conditions—1) Serum-free media and 2) Serum containing media. After treatment, we used JC 1 dye to study mitochondrial membrane potential (the dye fluoresces red in active mitochondria but green when mitochondria are compromised). The cells with more active mitochondria would be in better health than that with compromised mitochondria. The equal volume saline was used as a control. Fluorescence confocal microscopy imaging was done on live cells. Sovateltide treatment showed more mitochondrial protection than control in the serum-free condition.

The membrane-permeant JC-1 dye is widely used in apoptosis studies to monitor mitochondrial health. JC-1 dye can be used as an indicator of mitochondrial membrane potential in a variety of cell types, including myocytes and neurons, as well as in intact tissues and isolated mitochondria.

JC-1 is a novel cationic carbocyanine dye that accumulates in mitochondria. The dye exists as a monomer at low concentrations and yields green fluorescence, similar to fluorescein. At higher concentrations, the dye forms J-aggregates that exhibit a broad excitation spectrum and an emission maximum at ˜590 nm.

The cultured mouse cochlear cells were treated with 1 ng/ml IRL-1620 for 7 days in 2 different conditions—1) Serum free media and 2) Serum containing media. After treatment, we used JC 1 dye to study mitochondrial membrane potential (the dye fluoresces red in active mitochondria but green when mitochondria are compromised), which will indicate cellular health.

### DISCUSSION/CONCLUSIONS

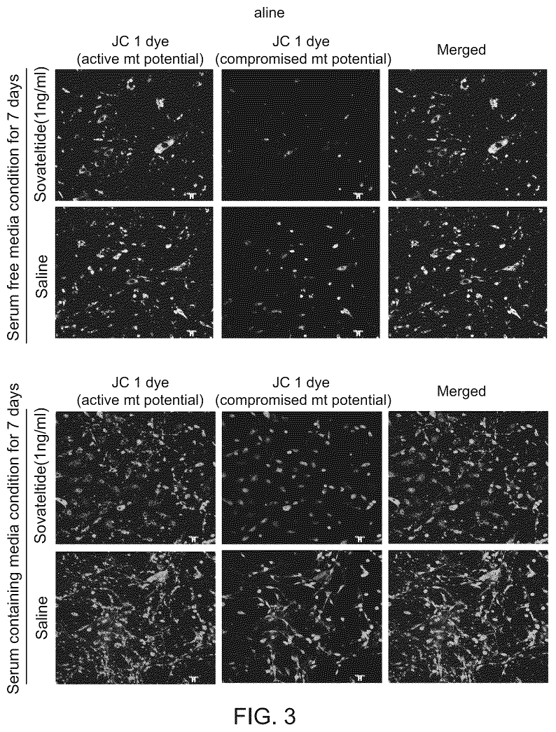

The cells with more number of active mitochondria will be in better health than that of compromised mitochondria. The equal volume saline was used as a control. At the conclusion of the experiment, imaging was performed on live cells, as shown in FIG. 3. Based on these images, we conclude that sovateltide treated cochlear cell cultures showed a notable improvement in mitochondrial protection and improved cellular viability in a serum-free condition where the damaging effect is more. The level of protection in this media was significant, as demonstrated by the reduction in compromised mitochondria. Serum containing medium did show a damaging effect on the mitochondria; however, it did demonstrate a reduction in compromised mitochondrial protection in sovateltide treated cultures.

Although the field of the present disclosure has been described herein with limited reference to specific embodiments, this description is not meant to be construed in a limiting sense. Various modifications of the disclosed embodiments, as well as alternate embodiments of the present disclosure, will become apparent to persons skilled in the art upon reference to the description of the disclosed embodiments.

### REFERENCES

ADAMSKY S and FEINSTEIN E (2013) Double-stranded RNA combination therapy targeting genes HES1, HES5, HEY2, CDKN1B, and NOTCH1, for use in treating hearing and balance disorders, p 110 pp., Quark Pharmaceuticals, Inc., USA.
ADAMSKY S and FEINSTEIN E (2014) Combination therapy for treating hearing and balance disorders, p No pp. given, Quark Pharmaceuticals, Inc., USA.
AKIYAMA N, YAMAMOTO-FUKUDA T, TAKAHASHI H and KOJI T: In situ tissue engineering with synthetic self-assembling peptide nanofiber scaffolds, PuraMatrix, for mucosal regeneration in the rat middle-ear. Int J Nanomedicine 8:2629-2640, 2013.
ALMEIDA-BRANCO M S, CABRERA S and LOPEZ-ESCAMEZ J A: Perspectives for the treatment of sensorineural hearing loss by cellular regeneration of the inner ear. Acta Otorrinolaringol Esp 66:286-295, 2015.
ARAI H, HORI S, ARAMORI I, OHKUBO H and NAKANISHI S: Cloning and expression of a cDNA encoding an endothelin receptor. Nature 348:730-732, 1990.
ARIANA B, GEERSE S, SCHOT L J and BOS L D: Modeled Analysis of Entrance of Colloid Suspensions into the Middle Ear Cavity. Otolaryngol Head Neck Surg 154:917-919, 2016.
BARNABE-HEIDER F, GORITZ C, SABELSTROM H, TAKEBAYASHI H, PFRIEGER F W, MELETIS K and FRISEN J: Origin of new glial cells in intact and injured adult spinal cord. Cell Stem Cell 7:470-482, 2010.
BAUER M, BAUER I, SONIN N V, KRESGE N, BAVEJA R, YOKOYAMA Y, HARDING D, ZHANG J X and CLEMENS M G: Functional significance of endothelin B receptors in mediating sinusoidal and extrasinusoidal effects of endothelins in the intact rat liver. Hepatology 31:937-947, 2000.
BORENSTEIN J T, PARARAS E, KIM E S, TANDON V, AYOOB A, MCKENNA M, SEWELL W, PEPPI M, WEINBERG M and LANGER R (2017) Method and process for quantitative evaluation of pharmacokinetics, therapeutic effects and safety of delivery of drug compounds to the inner ear for treatment of auditory diseases, p 51 pp., The Charles Stark Draper Laboratory Inc., USA; Massachusetts Eye and Ear Infirmary; Massachusetts Institute of Technology.
BRADLEY D J, TOWLE H C and YOUNG W S, 3R D: Alpha and beta thyroid hormone receptor (TR) gene expression during auditory neurogenesis: evidence for T R isoform-specific transcriptional regulation in vivo. Proc Natl Acad Sci USA 91:439-443, 1994.
BRAMHALL N F, SHI F, ARNOLD K, HOCHEDLINGER K and EDGE A S: Lgr5-positive supporting cells generate new hair cells in the postnatal cochlea. Stem Cell Reports 2:311-322, 2014.
BRAND M, L E MOULLEC J M, CORVOL P and GASC J M: Ontogeny of endothelins-1 and -3, their receptors, and endothelin converting enzyme-1 in the early human embryo. J Clin Invest 101:549-559, 1998.
BRIYAL S, NGUYEN C, LEONARD M and GULATI A: Stimulation of endothelin B receptors by IRL-1620 decreases the progression of Alzheimer's disease. Neuroscience 301:1-11, 2015.
BRIYAL S, SHEPARD C and GULATI A: Endothelin receptor type B agonist, IRL-1620, prevents beta amyloid (Abeta) induced oxidative stress and cognitive impairment in normal and diabetic rats. Pharmacology, biochemistry, and behavior 120:65-72, 2014.
BROOKS D P, DEPALMA P D, PULLEN M, GELLAI M and NAMBI P: Identification and function of putative ETB receptor subtypes in the dog kidney. J Cardiovasc Pharmacol 26 Suppl 3: S322-325, 1995.
BURNS J C and JACKSON J D (2013) Regeneration of inner ear cells that comprises administering an agent that increases the expression of Myc family proteins, p 83 pp., Wake Forest University Health Sciences, USA.
CHEN Z-Y (2014) Methods and compositions for regenerating inner ear hair cells and/or supporting cells using agents that increase c-myc or Notch activity, p 118 pp., Massachusetts Eye and Ear Infirmary, USA.
CHOI B Y, SONG J J, CHANG S O, KIM S U and OH S H: Intravenous administration of human mesenchymal stem cells after noise- or drug-induced hearing loss in rats. Acta Otolaryngol 132 Suppl 1: S94-102, 2012.
CIORBA A, ZATTARA S, LORONI G and PROSSER S: Quantitative enhancement of speech in noise through a wireless equipped hearing aid. Acta Otorhinolaryngol Ital 34:50-53, 2014.
DAVENPORT A P: International Union of Pharmacology. XXIX. Update on endothelin receptor nomenclature. Pharmacol Rev 54:219-226, 2002.
DAVENPORT A P, HYNDMAN K A, DHAUN N, SOUTHAN C, KOHAN D E, POLLOCK J S, POLLOCK D M, WEBB D J and MAGUIRE J J: Endothelin. Pharmacol Rev 68:357-418, 2016.
DEMBOWSKI C, HOFMANN P, KOCH T, KAMROWSKI-KRUCK H, RIEDESEL H, KRAMMER H J, KAUP F J and EHRENREICH H: Phenotype, intestinal morphology, and survival of homozygous and heterozygous endothelin B receptor-deficient (spotting lethal) rats. J Pediatr Surg 35:480-488, 2000.
DRUCKENBROD N R, POWERS P A, BARTLEY C R, WALKER J W and EPSTEIN M L: Targeting of endothelin receptor-B to the neural crest. Genesis 46:396-400, 2008.
DUSSAULT J H and RUEL J: Thyroid hormones and brain development. Annu Rev Physiol 49:321-334, 1987.
EHRENREICH H, NAU T R, DEMBOWSKI C, HASSELBLATT M, BARTH M, HAHN A, SCHILLING L, SIREN A L and BRUCK W: Endothelin b receptor deficiency is associated with an increased rate of neuronal apoptosis in the dentate gyrus. Neuroscience 95:993-1001, 2000.
EHRENREICH H, OLDENBURG J, HASSELBLATT M, HERMS J, DEMBOWSKI C, LOFFLER B M, BRUCK W, KAMROWSKI-KRUCK H, GALL S, SIREN A L and SCHILLING L: Endothelin B receptor-deficient rats as a subtraction model to study the cerebral endothelin system. Neuroscience 91:1067-1075, 1999.
E L KECHAI N, MAMELLE E, NGUYEN Y, HUANG N, NICOLAS V, CHAMINADE P, YEN-NICOLAY S, GUEUTIN C, GRANGER B, FERRARY E, AGNELY F and BOCHOT A: Hyaluronic acid liposomal gel sustains delivery of a corticoid to the inner ear. J Control Release 226:248-257, 2016.
ENGEBRETSON A M and FREDRICKSON J (1992) Implantable hearing aid coupler device, Google patents.
ERIKSSON P S, PERFILIEVA E, BJORK-ERIKSSON T, ALBORN A M, NORDBORG C, PETERSON D A and GAGE F H: Neurogenesis in the adult human hippocampus. Nat Med 4:1313-1317, 1998.
FARSETTI A, MITSUHASHI T, DESVERGNE B, ROBBINS J and NIKODEM V M: Molecular basis of thyroid hormone regulation of myelin basic protein gene expression in rodent brain. J Biol Chem 266:23226-23232, 1991.
FELLNER S K and ARENDSHORST W: Endothelin-A and -B receptors, superoxide, and Ca2+ signaling in afferent arterioles. Am J Physiol Renal Physiol 292: F175-184, 2007.
FILIPO R, ATTANASIO G, RUSSO F Y, CARTOCCI G, MUSACCHIO A, D E CARLO A, ROUKOS R, D E SETA E, DI TILLO G, VICCARO M, SARNACCHIARO P and COVELLI E: Oral versus short-term intratympanic prednisolone therapy for idiopathic sudden hearing loss. Audiol Neurootol 19:225-233, 2014.
FLORES-OTERO J and DAVIS R L: Synaptic proteins are tonotopically graded in postnatal and adult type I and type II spiral ganglion neurons. J Comp Neurol 519:1455-1475, 2011.
GELLAI M, FLETCHER T, PULLEN M and NAMBI P: Evidence for the existence of endothelin-B receptor subtypes and their physiological roles in the rat. Am J Physiol 271: R254-261, 1996.
GUL, CUI X, WEI W, YANG J and LI X: Ferulic acid promotes survival and differentiation of neural stem cells to prevent gentamicin-induced neuronal hearing loss. Exp Cell Res 360:257-263, 2017.
GULATI A: Endothelin Receptors, Mitochondria and Neurogenesis in Cerebral Ischemia. Curr Neuropharmacol 14:619-626, 2016.
GULATI A, KUMAR A, MORRISON S and SHAHANI B T: Effect of centrally administered endothelin agonists on systemic and regional blood circulation in the rat: role of sympathetic nervous system. Neuropeptides 31:301-309, 1997.
GULATI A, KUMAR A and SHAHANI B T: Cardiovascular effects of centrally administered endothelin-1 and its relationship to changes in cerebral blood flow. Life Sci 58:437-445, 1996.
GULATI A, REBELLO S, CHARI G and BHAT R: Ontogeny of endothelin and its receptors in rat brain. Life Sci 51:1715-1724, 1992.
GULATI A, REBELLO S, ROY S and SAXENA P R: Cardiovascular effects of centrally administered endothelin-1 in rats. J Cardiovasc Pharmacol 26 Suppl 3: S244-246, 1995.
GULATI A and SRIMAL R C: Endothelin antagonizes the hypotension and potentiates the hypertension induced by clonidine. Eur J Pharmacol 230:293-300, 1993.
GULATI S, BRIYAL S, JONES S, BHALLA S and GULATI A: Attenuation of opioid tolerance by ETB receptor agonist, IRL-1620, is independent of an accompanied decrease in nerve growth factor in mice. Heliyon 3: e00317, 2017.
HÅKANSSON B (2005) Percutaneous bone anchored transferring device, Google patents.
HAN Z, WANG C P, CONG N, GU Y Y, MA R and CHI F L: Therapeutic value of nerve growth factor in promoting neural stem cell survival and differentiation and protecting against neuronal hearing loss. Mol Cell Biochem 428:149-159, 2017.
HANSEN M R, ZHA X M, BOK J and GREEN S H: Multiple distinct signal pathways, including an autocrine neurotrophic mechanism, contribute to the survival-promoting effect of depolarization on spiral ganglion neurons in vitro. J Neurosci 21:2256-2267, 2001.
HARLAND S P, KUC R E, PICKARD J D and DAVENPORT A P: Characterization of endothelin receptors in human brain cortex, gliomas, and meningiomas. J Cardiovasc Pharmacol 26 Suppl 3: S408-411, 1995.
HEIDE J and PRESCOTT A D (1993) Trans-tympanic connector for magnetic induction hearing aid, Google patents.
HILDEBRAND M S, DAHL H H, HARDMAN J, COLEMAN B, SHEPHERD R K and D E SILVA M G: Survival of partially differentiated mouse embryonic stem cells in the scala media of the guinea pig cochlea. J Assoc Res Otolaryngol 6:341-354, 2005.
HIROSE K, DISCOLO C M, KEASLER J R and RANSOHOFF R: Mononuclear phagocytes migrate into the murine cochlea after acoustic trauma. J Comp Neurol 489:180-194, 2005.
HIROSE K and LIBERMAN M C: Lateral wall histopathology and endocochlear potential in the noise-damaged mouse cochlea. J Assoc Res Otolaryngol 4:339-352, 2003.
HOSOYA M, OKANO H and FUJIOKA M (2015) Inner ear cell induction method applied in regenerative medicine or research tool, p 16 pp., Keio University, Japan.
HOSTENBACH S, D'HAESELEER M, KOOIJMAN R and DE KEYSER J: The pathophysiological role of astrocytic endothelin-1. Prog Neurobiol 144:88-102, 2016.
HUANG X, CHEN M, DING Y and WANG Q: Arctigenin protects against neuronal hearing loss by promoting neural stem cell survival and differentiation. Genesis 55, 2017.
INAOKA T, NAKAGAWA T, KIKKAWA Y S, TABATA Y, ONO K, YOSHIDA M, TSUBOUCHI H, IDO A and ITO J: Local application of hepatocyte growth factor using gelatin hydrogels attenuates noise-induced hearing loss in guinea pigs. Acta Otolaryngol 129:453-457, 2009.
INOUE A, YANAGISAWA M, KIMURA S, KASUYA Y, MIYAUCHI T, GOTO K and MASAKI T: The human endothelin family: three structurally and pharmacologically distinct isopeptides predicted by three separate genes. Proc Natl Acad Sci USA 86:2863-2867, 1989.
JULSTROM S D, DRAMBAREAN V and SOEDE W (2009) Multi-coil coupling system for hearing aid applications, Google patents.
KAMIYA K, FUJINAMI Y, HOYA N, OKAMOTO Y, KOUIKE H, KOMATSUZAKI R, KUSANO R, NAKAGAWA S, SATOH H, FUJII M and MATSUNAGA T: Mesenchymal stem cell transplantation accelerates hearing recovery through the repair of injured cochlear fibrocytes. Am J Pathol 171:214-226, 2007.
KANG W S, SUN S, NGUYEN K, KASHEMIROV B, MCKENNA C E, HACKING S A, QUESNEL A M, SEWELL W F, MCKENNA M J and JUNG D H: Non-Ototoxic Local Delivery of Bisphosphonate to the Mammalian Cochlea. Otol Neurotol 36:953-960, 2015.
KASAGI H, KUHARA T, OKADA H, SUEYOSHI N and KURIHARA H: Mesenchymal stem cell transplantation to the mouse cochlea as a treatment for childhood sensorineural hearing loss. Int J Pediatr Otorhinolaryngol 77:936-942, 2013.
KATO M, OGAMI N, ITA M and TAGUCHI N (2011) Age-related or noise-induced hearing loss and tinnitus prophylactic/therapeutic agents containing endothelin B receptor expression promoters, compositions containing the agents, production of transient hearing loss mouse model, use thereof, drug screening using the mouse, and detection or prediction of the hearing disorders, p 56 pp., Chubu University, Japan; Nagoya University.
KAUNDAL R K, DESHPANDE T A, GULATI A and SHARMA S S: Targeting endothelin receptors for pharmacotherapy of ischemic stroke: current scenario and future perspectives. Drug discovery today 17:793-804, 2012.
KHAN H, NAYLOR R J and TULADHAR B R: Pharmacological characterization of endothelin receptors-mediated contraction in the mouse isolated proximal and distal colon. Br J Pharmacol 147:607-611, 2006.
KUHN H G, DICKINSON-ANSON H and GAGE F H: Neurogenesis in the dentate gyrus of the adult rat: age-related decrease of neuronal progenitor proliferation. J Neurosci 16:2027-2033, 1996.
LAWRENCE E, SINEY L, WILSONCROFT P, KNOCK G A, TERENGHI G, POLAK J M and BRAIN S D: Evidence for ETA and ETB receptors in rat skin and an investigation of their function in the cutaneous microvasculature. Br J Pharmacol 115:840-844, 1995.
LEE K Y, NAKAGAWA T, OKANO T, HORI R, ONO K, TABATA Y, LEE S H and ITO J: Novel therapy for hearing loss: delivery of insulin-like growth factor 1 to the cochlea using gelatin hydrogel. Otol Neurotol 28:976-981, 2007.
LEONARD M G, BRIYAL S and GULATI A: Endothelin B receptor agonist, IRL-1620, reduces neurological damage following permanent middle cerebral artery occlusion in rats. Brain Res 1420:48-58, 2011.
LEONARD M G, BRIYAL S and GULATI A: Endothelin B receptor agonist, IRL-1620, provides long-term neuroprotection in cerebral ischemia in rats. Brain Res 1464:14-23, 2012.
LEONARD M G and GULATI A: Endothelin B receptor agonist, IRL-1620, enhances angiogenesis and neurogenesis following cerebral ischemia in rats. Brain Res 1528:28-41, 2013.
LEONARD M G, PRAZAD P, PUPPALA B and GULATI A: Selective Endothelin-B Receptor Stimulation Increases Vascular Endothelial Growth Factor in the Rat Brain during Postnatal Development. Drug Res (Stuttg) 65:607-613, 2015.
LI L, CHAO T, BRANT J, O'MALLEY B, JR., TSOURKAS A and LI D: Advances in nano-based inner ear delivery systems for the treatment of sensorineural hearing loss. Adv Drug Deliv Rev 108:2-12, 2017.
LI M L, LEE L C, CHENG Y R, KUO C H, CHOU Y F, CHEN Y S, YAO C M, CHEN P R, HSU C J, SONG Y L and LEE C F: A novel aerosol-mediated drug delivery system for inner ear therapy: intratympanic aerosol methylprednisolone can attenuate acoustic trauma. IEEE Trans Biomed Eng 60:2450-2460, 2013.
LIU Q, FENG B, MENG G and LIU Y: [The study on plasma ET and NO of patients with sudden hearing loss]. Lin Chuang Er Bi Yan Hou Ke Za Zhi 17:668-669, 2003.
MACCUMBER M W, ROSS C A and SNYDER S H: Endothelin in brain: receptors, mitogenesis, and biosynthesis in glial cells. Proc Natl Acad Sci USA 87:2359-2363, 1990.
MARTINI A, CASTIGLIONE A, BOVO R, VALLESI A and GABELLI C: Aging, cognitive load, dementia and hearing loss. Audiol Neurootol 19 Suppl 1:2-5, 2014.
MATHISON Y and ISRAEL A: Endothelin E T (B) receptor subtype mediates nitric oxide/cGMP formation in rat adrenal medulla. Brain Res Bull 45:15-19, 1998.
MAZZONI M R, BRESCHI M C, CECCARELLI F, LAZZERI N, GIUSTI L, NIERI P and LUCACCHINI A: Suc-[Glu9,Ala11,15]-endothelin-1 (8-21), IRL 1620, identifies two populations of E T (B) receptors in guinea-pig bronchus. Br J Pharmacol 127:1406-1414, 1999.
MCLEAN W (2017) Methods for controlled proliferation of vestibular stem cells/generating inner ear hair cells using Wnt and TGF-β inhibition, p 93 pp., Frequency Therapeutics, Inc., USA.
MIASIRO N, KARAKI H and PAIVA A C: Distinct endothelin-B receptors mediate the effects of sarafotoxin S6c and IRL1620 in the ileum. J Cardiovasc Pharmacol 31 Suppl 1: S175-178, 1998.
MILLER J M, ALTSCHULER R A and RAPHAEL Y (2010) Auditory nerve protection and re-growth, Google patents.
MONEY D, NYGARD T, SELIGMAN P, IBRAHIM I and ZHANG A L (2008) Cochlear implant, Google patents.
MULLER U and BARR-GILLESPIE P G: New treatment options for hearing loss. Nat Rev Drug Discov 14:346-365, 2015.
NICKEL R and FORGE A: Gap junctions and connexins in the inner ear: their roles in homeostasis and deafness. Curr Opin Otolaryngol Head Neck Surg 16:452-457, 2008.
OKANO T and KELLEY M W: Stem cell therapy for the inner ear: recent advances and future directions. Trends Amplif 16:4-18, 2012.
OSHIMA K, SHIN K, DIENSTHUBER M, PENG A W, RICCI A J and HELLER S: Mechanosensitive hair cell-like cells from embryonic and induced pluripotent stem cells. Cell 141:704-716, 2010.
OTTONE C, KRUSCHE B, WHITBY A, CLEMENTS M, QUADRATO G, PITULESCU M E, ADAMS R H and PARRINELLO S: Direct cell-cell contact with the vascular niche maintains quiescent neural stem cells. Nat Cell Biol 16:1045-1056, 2014.
PARRA A, PONTE C, CENJOR C, GARCIA-CALVO G, GIMENEZ M J, AGUILAR L and SORIANO F: Optimal dose of amoxicillin in treatment of otitis media caused by a penicillin-resistant pneumococcus strain in the gerbil model. Antimicrob Agents Chemother 46:859-862, 2002.
PUPPALA B, AWAN I, BRIYAL S, MBACHU O, LEONARD M and GULATI A: Ontogeny of endothelin receptors in the brain, heart, and kidneys of neonatal rats. Brain Dev 37:206-215, 2015.
QUESNEL A M, SETON M, MERCHANT S N, HALPIN C and MCKENNA M J: Third-generation bisphosphonates for treatment of sensorineural hearing loss in otosclerosis. Otol Neurotol 33:1308-1314, 2012.
RIVOLTA M N: New strategies for the restoration of hearing loss: challenges and opportunities. Br Med Bull 105:69-84, 2013.
RODRIGUEZ-PENA A, IBARROLA N, INIGUEZ M A, MUNOZ A and BERNAL J: Neonatal hypothyroidism affects the timely expression of myelin-associated glycoprotein in the rat brain. J Clin Invest 91:812-818, 1993.
ROELOFS A J, COXON F P, EBETINO F H, LUNDY M W, HENNEMAN Z J, NANCOLLAS G H, SUN S, BLAZEWSKA K M, BALA J L, KASHEMIROV B A, KHALID A B, MCKENNA C E and ROGERS M J: Fluorescent risedronate analogues reveal bisphosphonate uptake by bone marrow monocytes and localization around osteocytes in vivo. J Bone Miner Res 25:606-616, 2010.
SAMEER MALLICK A, QUREISHI A, PEARSON R and O′DONOGHUE G: Neurotrophins and cochlear implants: a solution to sensorineural deafness? Cochlear implants international 14:158-164, 2013.
SCHACHT J, TALASKA A E and RYBAK L P: Cisplatin and aminoglycoside antibiotics: hearing loss and its prevention. Anat Rec (Hoboken) 295:1837-1850, 2012.
SCHERER E Q, ARNOLD W and WANGEMANN P: Pharmacological reversal of endothelin-1 mediated constriction of the spiral modiolar artery: a potential new treatment for sudden sensorineural hearing loss. BMC Ear Nose Throat Disord 5:10, 2005.
SCHERER E Q, WONNEBERGER K and WANGEMANN P: Differential desensitization of Ca2+ mobilization and vasoconstriction by E T (A) receptors in the gerbil spiral modiolar artery. J Membr Biol 182:183-191, 2001.
SCHINELLI S: Pharmacology and physiopathology of the brain endothelin system: an overview. Current medicinal chemistry 13:627-638, 2006.
SEIXAS N S, NEITZEL R, STOVER B, SHEPPARD L, FEENEY P, MILLS D and KUJAWA S: 10-Year prospective study of noise exposure and hearing damage among construction workers. Occup Environ Med 69:643-650, 2012.
SIMONI E, ORSINI G, CHICCA M, BETTINI S, FRANCESCHINI V, MARTINI A and ASTOLFI L: Regenerative medicine in hearing recovery. Cytotherapy 19:909-915, 2017.
SOKOLOVSKY M, AMBAR I and GALRON R: A novel subtype of endothelin receptors. J Biol Chem 267:20551-20554, 1992.
SPICER S S and SCHULTE B A: Spiral ligament pathology in quiet-aged gerbils. Hear Res 172:172-185, 2002.
STANKOVIC K, RIO C, XIA A, SUGAWARA M, ADAMS J C, LIBERMAN M C and CORFAS G: Survival of adult spiral ganglion neurons requires erbB receptor signaling in the inner ear. J Neurosci 24:8651-8661, 2004.
STANKOVIC K M and CORFAS G: Real-time quantitative R T-PCR for low-abundance transcripts in the inner ear: analysis of neurotrophic factor expression. Hear Res 185:97-108, 2003.
STRENZKE N, PAULI-MAGNUS D, MEYER A, BRANDT A, MAIER H and MOSER T: [Update on physiology and pathophysiology of the inner ear: pathomechanisms of sensorineural hearing loss]. HNO 56:27-36, 2008.
SUGAWARA M, MURTIE J C, STANKOVIC K M, LIBERMAN M C and CORFAS G: Dynamic patterns of neurotrophin 3 expression in the postnatal mouse inner ear. J Comp Neurol 501:30-37, 2007.
SUN S, BLAZEWSKA K M, KADINA A P, KASHEMIROV B A, DUAN X, TRIFFITT J T, DUNFORD J E, RUSSELL R G, EBETINO F H, ROELOFS A J, COXON F P, LUNDY M W and MCKENNA C E: Fluorescent Bisphosphonate and Carboxyphosphonate Probes: A Versatile Imaging Toolkit for Applications in Bone Biology and Biomedicine. Bioconjug Chem 27:329-340, 2016.
SWAN E E, MESCHER M J, SEWELL W F, TAO S L and BORENSTEIN J T: Inner ear drug delivery for auditory applications. Adv Drug Deliv Rev 60:1583-1599, 2008.
TAKAI M, UMEMURA I, YAMASAKI K, WATAKABE T, FUJITANI Y, ODA K, URADE Y, INUI T, YAMAMURA T and OKADA T: A potent and specific agonist, Suc-[Glu9, Ala11,15]-endothelin-1 (8-21), IRL 1620, for the ET_{B} receptor. Biochem Biophys Res Commun 184:953-959, 1992.
TROWE M O, MAIER H, SCHWEIZER M and KISPERT A: Deafness in mice lacking the T-box transcription factor Tbx18 in otic fibrocytes. Development 135:1725-1734, 2008.
TUYSUZ B, COLLIN A, ARAPOGLU M and SUYUGUL N: Clinical variability of Waardenburg-Shah syndrome in patients with proximal 13q deletion syndrome including the endothelin-B receptor locus. Am J Med Genet A 149A: 2290-2295, 2009.
UCHIDA Y, TERANISHI M, NISHIO N, SUGIURA S, HIRAMATSU M, SUZUKI H, KATO K, OTAKE H, YOSHIDA T, TAGAYA M, SUZUKI H, SONE M, ANDO F, SHIMOKATA H and NAKASHIMA T: Endothelin-1 gene polymorphism in sudden sensorineural hearing loss. Laryngoscope 123: E59-65, 2013.
VALENTE F, ASTOLFI L, SIMONI E, DANTI S, FRANCESCHINI V, CHICCA M and MARTINI A: Nanoparticle drug delivery systems for inner ear therapy: an overview. Journal of Drug Delivery Science and Technology 39:28-35, 2017.
VIDOVIC M, CHEN M M, LU Q Y, KALLONIATIS K F, MARTIN B M, TAN A H, LYNCH C, CROAKER G D, CASS D T and SONG Z M: Deficiency in endothelin receptor B reduces proliferation of neuronal progenitors and increases apoptosis in postnatal rat cerebellum. Cell Mol Neurobiol 28:1129-1138, 2008.
VILLAR-FERNANDEZ M A and LOPEZ-ESCAMEZ J A: Outlook for Tissue Engineering of the Tympanic Membrane. Audiol Res 5:117, 2015.
VITELLI F, VIOLA A, MORISHIMA M, PRAMPARO T, BALDINI A and LINDSAY E: TBX1 is required for inner ear morphogenesis. Hum Mol Genet 12:2041-2048, 2003.
WALLACE G G, HIGGINS M J, MOULTON S E and WANG C: Nanobionics: the impact of nanotechnology on implantable medical bionic devices. Nanoscale 4:4327-4347, 2012.
WEST M B and KOPKE R D (2017) Combination therapies for inner ear sensory hair cell regeneration/replacement using an agent that decreases expression of Hes1 gene such as siRNA and a second agent, p 98 pp., Hough Ear Institute, USA.
WINTERSTEIN A G, LIU W, X U D and ANTONELLI P J: Sensorineural hearing loss associated with neomycin eardrops and nonintact tympanic membranes. Otolaryngol Head Neck Surg 148:277-283, 2013.
XU X N, HUANG J M, LIN G J and JIANG Z Z: [Contributions of endothelin in the process of the noise-induced injury of inner ear]. Zhonghua Er Bi Yan Hou Tou Jing Wai Ke Za Zhi 40:509-512, 2005.
YANAGISAWA M, KURIHARA H, KIMURA S, TOMOBE Y, KOBAYASHI M, MITSUI Y, YAZAKI Y, GOTO K and MASAKI T: A novel potent vasoconstrictor peptide produced by vascular endothelial cells. Nature 332:411-415, 1988.
YANG C H, HUANG K S, WANG C Y, HSU Y Y, CHANG F R and LIN Y S: Microfluidic-assisted synthesis of hemispherical and discoidal chitosan microparticles at an oil/water interface. Electrophoresis 33:3173-3180, 2012.
ZHENG H, TAI H and ZENG S: Cochlear stem cell transplantation for hearing recovery in a rat model of sensorineural hearing loss**★. Neural Regeneration Research 5:833-837, 2010.
ZHURAVLEV D A (2017) Agent for sensorineural hearing loss treatment, p 18 pp., Russia.

What is claimed is:

1. A pharmaceutical composition comprising, endothelin B receptor analogs selected from the group consisting of N-Succinyl-[Glu⁹, Ala^{11,15}] endothelin 1 (IRL-1620, sovateltide), BQ-3020, [Ala^{1,3,11,15}]-endothelin, sarafotoxin S6c, endothelin-3 and a mixture thereof.

2. The pharmaceutical composition of claim 1, wherein the vehicle for endothelin B receptor analogs administration is via a sustained release drug delivery system such as Gelfoam®, Hyaluronic gel, Seprapack™, Thiol-modified Hyaluronic acid, Glutaradehyde cross-linking of porcine type-collagen, gelatin, chitosan glycosylated derivative, Poloxamer 407, chitosan glycerophosphate hydrogel, sustained release are Lipid core nanocapsules poly L-lysine (HBPL) nanoparticles.

3. The pharmaceutical composition of claim 1, wherein endothelin analog dose range is 0.00001 to about 1 mg.

4. A method of subject for treating sensorineural hearing loss and vestibular symptoms caused by degeneration of cells of the inner ear using a compound that includes endothelin analogs by the mechanism of neuroprotection and neurogenesis of cochleovestibular hair cells, support cells, and synapses.

5. The method of claim 1, wherein said cochleovestibular hair cells, support cells, and synapses are spiral ganglion neurons, and/or inner hair cells and/or outer hair cells.

6. The method of claim 1 comprising the pharmaceutical composition of claim 1 may be used with an additional agent such as growth factors, or osmotic agent such as glycerol, or diuretic such as furosemide or ethacrynic acid, or a steroid, or transplanted stem cells, or ferulic acid, or arctigenin, or espin isoform 1 polypeptide, or neurotrophin selected from the group consisting of glial cell line-derived neurotrophic factor, brain-derived neurotrophic factor, neurotrophin-3, neurotrophin-4/5, and ciliary neurotrophic factor, or human insulin-like growth factor 1, or Netrin1, or a mixture thereof, and optionally an excipient.

7. The method of claim 1, wherein endothelin B receptor analogs are delivered intratympanically by procedures that rely on diffusion through middle ear barriers for entry into the cochleovestibular apparatus. Procedures could be through Silverstein Microwick, or through a round window microcatheter, or through intratympanic injection using a hollow bore needle.

8. The method of claim 1, wherein endothelin B receptor analogs are delivered intracochlear by procedures through a cochleostomy, direct injections, osmotic mini-pumps, and reciprocating perfusion systems.

9. The method of claim 1, wherein endothelin B receptor analogs are delivered intravestibular by procedures through the endolymphatic sac, endolymphatic duct.

10. The method of claim 1, wherein endothelin B receptor analogs are delivered intravenously, orally, intramuscularly, and subcutaneously.

11. The method of claim 1, wherein endothelin B receptor analogs are delivered intracochlear while coated on a cochlear implantable device.

12. The method of claim 1, wherein endothelin B receptor analogs are conjugated with nanoparticles, including stealth nanoparticles and hydrogels.

13. The method of the subject where the pharmaceutical composition in claim 1 is used to treat disequilibrium, aural fullness, tinnitus, fluctuating sensorineural hearing loss, sudden sensorineural hearing loss, noise-induced hearing loss, vestibular neuritis, Meniere's disease, labyrinthitis, medication-induced sensorineural hearing loss, sensorineural hearing loss due to a genetic condition, congenital sensorineural hearing loss, auditory neuropathy.

14. The method of claim 1, wherein endothelin analogs administration may be combined or used with an additional agent such as growth factors, or osmotic agents such as glycerol, or diuretic such as furosemide or ethacrynic acid, or a steroid, or transplanted stem cells, or ferulic acid, or arctigenin, or espin isoform 1 polypeptide, or neurotrophin selected from the group consisting of glial cell line-derived neurotrophic factor, brain-derived neurotrophic factor, neurotrophin-3, neurotrophin-4/5, and ciliary neurotrophic factor, or human insulin-like growth factor 1, or Netrin1, or a mixture thereof.

15. An article of manufacture comprising:
(a) a packaged composition comprising an endothelin-B (ET_{B}) receptor agonist and steroid or a salt thereof;
(b) an insert providing instructions for a simultaneous or sequential administration of the ET_{B} receptor agonist and the steroid or salt thereof to treat a patient; and
(c) a container for (a) and (b).
